(19) United States
(12) Patent Application Publication
(10) Pub. No.: US 2024/0018460 A1
(43) Pub. Date: Jan. 18, 2024

(54) METHODS AND APPARATUS FOR SCALING IN BIOPROCESS SYSTEMS

(71) Applicant: Global Life Sciences Solutions USA LLC, Marlborough, MA (US)

(72) Inventors: Andreas CASTAN, Uppsala (SE); Nagaraju KONDURU, Bengaluru (IN); Ashish HANDA, Bengaluru (IN); Neelima BODDAPATI, Bengaluru (IN); Helena ÖHRVIK, Uppsala (SE); Alok Singh CHAUHAN, Bengaluru (IN)

(21) Appl. No.: 18/256,185

(22) PCT Filed: Sep. 29, 2021
(86) PCT No.: PCT/EP2021/076862

§ 371 Date: Jun. 6, 2023

(30) Foreign Application Priority Data
Dec. 7, 2020 (IN) 202011053215

(51) Int. Cl. C12M 1/36; C12M 1/00; C12M 1/34

(52) U.S. Cl. CPC C12M 41/48; C12M 29/06; C12M 23/40; C12M 41/46; C12M 41/34

(57) ABSTRACT

Methods and apparatus for scaling in bioprocess systems are disclosed. An example apparatus for bioprocess scaling includes at least one memory to store instructions, and processor circuitry to execute the instructions to identify an operating parameter of a target bioreactor, determine an upper boundary or a lower boundary defining a design space for at least one bioreactor process parameter to match at least one of a first target parameter range or a second target parameter range based on the operating parameter, simulate changes in the first target parameter range or a second target parameter range based on an adjustment to the upper boundary or the lower boundary in the design space, and configure the target bioreactor using output obtained from the adjustment to the upper boundary or the lower boundary to identify a match between the first target parameter range or the second target parameter range.

### RELATED APPLICATIONS

This patent arises from the national stage of International Application No. PCT/EP21/076862, which was filed on Sep. 29, 2021, which claims priority to Indian Provisional Application 202011053215, which was filed on Dec. 7, 2020. Indian Provisional Application 202011053215 and International Application No. PCT/EP21/076862 are hereby incorporated herein by reference in their entirety. Priority to Indian Provisional Application 202011053215 and International Application No. PCT/EP21/076862 is hereby claimed.

### FIELD OF THE DISCLOSURE

This disclosure relates generally to bioprocess systems and, more particularly, to methods and apparatus for scaling in bioprocess systems.

### BACKGROUND

Bioprocesses are used to produce medically and industrially critical products (e.g., therapeutics, biofuels, etc.) using biomanufacturing through optimization of natural and/or artificial biological systems to allow for large-scale production. Instruments for bioprocess control and analysis are used for maintaining optimal environmental conditions by monitoring and controlling operational variables (e.g., flow rate, temperature, pH, pressure, agitator shaft power, rate of stifling, etc.). As such, physical, chemical, and biological parameters must be kept constant or maintained at optimal levels to prevent any deviations from a set range.

### BRIEF DESCRIPTION OF THE DRAWINGS

FIG. 1A illustrates an example operating window for aeration and agitation in bioprocess systems.

FIG. 1B illustrates example bioreactor scale-up parameters that can be kept constant during bioreactor scaling.

FIG. 2 is a block diagram illustrating an example environment for bioreactor scaling in accordance with the teachings of this disclosure.

FIG. 3 is a block diagram of an example implementation of the bioprocess scaler of FIG. 2 to facilitate bioreactor scaling.

FIG. 4 is a flowchart representative of example machine-readable instructions that may be executed during bioreactor scaling.

FIG. 5 is a flowchart representative of example machine-readable instructions that may be executed to determine set points during bioreactor scaling.

FIG. 6 illustrates an example user interface including a target bioreactor and a reference bioreactor and their associated target and reference scales, respectively.

FIG. 7 illustrates an example user interface including reference and standard values for multiple variables during bioreactor scaling.

FIG. 8 illustrates an example user interface including an example simulation functionality that gives a user the ability to explore a design space and find other settings that maintain desired criteria.

FIG. 9 illustrates an example design space configuration interface.

FIG. 10 illustrates an example input section indicating cell line information, reference scale information, target scale information, and parameter entry.

FIG. 11 illustrates an example graphical output showing results associated with variations in aeration, mixing time, agitation, working volume, tip speed, and/or primary sparger volumetric mass transfer coefficient (kLa).

FIG. 12 illustrates an example target process view, including a user-based option to show results associated with the target scale, the reference scale, and/or a combination of both the target scale and the reference scale.

FIG. 13 illustrates an example scale conversion tool and example scaling strategy options to reduce differences between the reference scale and the target scale.

FIG. 14 illustrates an example single day scaling and/or multi-day scaling display for viewing cell line information, reference and/or target bioreactor information, and/or the corresponding scaling strategy.

FIG. 15 illustrates an example selection of the scaling strategy options illustrated in FIG. 14.

FIG. 16 illustrates an example bioreactor detail user interface, including bioreactor information, configuration, and/or agitation selections.

FIG. 17A illustrates an example power input user interface, including selection of a power input model.

FIG. 17B illustrates example mixing time data user interface, including selection of a mixing time model.

FIG. 17C illustrates example volumetric mass transfer coefficient (kLa) details, including selection of a kLa model.

FIG. 18 illustrates an example review and create user interface, including a summary of the selections shown in FIGS. 16, 17A, 17B, and/or 17C.

FIG. 19 illustrates an example user interface showing results that can be viewed interactively based on the selection of a particular day of interest.

FIG. 20 illustrates an example user interface showing target process parameter data generation.

FIG. 21 is a block diagram of an example processing platform structured to execute the example instructions of FIGS. 4-5 to implement the example bioprocess scaler of FIGS. 2 and 3.

FIG. 22 is a block diagram of an example implementation of the processor circuitry of FIG. 21.

FIG. 23 is a block diagram of another example implementation of the processor circuitry of FIG. 21.

FIG. 24 is a block diagram of an example software distribution platform (e.g., one or more servers) to distribute software (e.g., software corresponding to the example machine readable instructions of FIG. 21) to client devices associated with end users and/or consumers (e.g., for license, sale, and/or use), retailers (e.g., for sale, re-sale, license, and/or sub-license), and/or original equipment manufacturers (OEMs) (e.g., for inclusion in products to be distributed to, for example, retailers and/or to other end users such as direct buy customers).

The figures are not scale. Wherever possible, the same reference numbers will be used throughout the drawings and accompanying written description to refer to the same or like parts.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

Bioprocesses requires real-time, continuous measurement of process variables to ensure the stability, efficiency, and reproducibility of the process to provide for a high-quality product. By measuring quality-related process variables that are necessary to maintain a narrow range of environmental conditions, consistent reproduction of the desired product can be achieved and documented. A variety of bioprocess instruments (also referred to herein as bioprocess units) are used during upstream processing (e.g., biomass expansion, media development and preparation, etc.) and downstream processing (e.g., product extraction and purification from the biomass, etc.), including bioreactors and mixers. For example, a bioreactor can be used to create a controlled environment for in vitro management of cells (e.g., cell proliferation, differentiation, etc.) during upstream processing. Bioreactors can include sensors directly interfacing, or used in conjunction with, the bioreactors to measure process variables, including oxygen and carbon dioxide concentration, biomass concentration, flow injection, and/or overall media composition.

Bioprocesses (e.g., use of living cells and/or cell components to obtain products such as biotherapeutics) can be developed at smaller scales before stepwise transfer to larger volumes occurs to achieve industrial production-scale levels (e.g., scaling up based on bioreactor operating parameters from a smaller scale to a larger scale the process is transferred to). Reliable bioprocess scaling up is needed to achieve consistent products of high quality, including high product yields. For example, mass transfer within a process can be highly dependent on a given scale, with bioreactor configuration (e.g., bioreactor geometry, impeller, etc.) influencing mixing times and resulting oxygen uptake, among other variables. Scale-up criteria can include consideration of parameters such as vessel and impeller geometry, tip speed, mixing time, oxygen transfer rate, and/or volumetric mass transfer coefficient (kLa). For example, a process parameter can be maintained constant throughout the scale-up process to reduce any negative effects of changes in the bioprocess environment. In some examples, the parameters that are altered can depend on the type of bioprocess (e.g., cultures with cells sensitive to high shear forces can have a set tip speed and/or an acceptable tip speed range). In some examples, oxygen can be a limiting factor for growth of a given culture, thereby requiring similar oxygen transfer rates (OTRs) between scales during scale-up and/or optimization of the mass transfer coefficient (kLa). Additionally, bioreactor impeller size and geometry can be selected based on a specific working volume to maintain a constant impeller-based power consumption per liquid volume (P/V). For example, scaling a process from a microbioreactor scale (e.g., 10 microliters) to production scale (e.g., 1000 Liters) requires consideration of multiple scaling criteria including physical operating parameters and their effect on product quality across various scales.

While various scaling methodologies can be used, it is important to consider the design space within which adjustments to parameters can be made without jeopardizing the integrity of the bioprocess itself. For example, scaling requires staying within the same design space across scales (e.g., when moving from a reference bioreactor where a small-scale bioreactor can be used to initially developed a bioprocess to a target bioreactor where the bioprocess is on a large scale production). Multiple relevant scaling parameters can make it difficult to define the optimal design space. Furthermore, once the design space is defined and upper and/or lower boundaries are set, a user may not be able to modify some parameters without having a clear indication of how the desired changes will affect the resulting parameters that will define the outcome of the bioprocess itself (e.g., oxygen transfer rate, etc.).

Methods and apparatus for scaling in bioprocess systems described herein permit the identification of a range of potential changes in bioprocess variables that can be optimized while staying within a given design space. For example, current approaches used for scaling include determining the scale being used to scale from (e.g., reference scale) and scaling to a target scale, with set point(s) extracted from the reference scale. During scaling, one or more scaling parameters (e.g., tip speed, energy dissipation rate, mixing time, mass transfer coefficient, turbulent shear forces, etc.) are selected to be maintained during scaling, with new set point(s) for the target scale determined to fulfill as many of the scaling parameters as possible. While this approach includes projecting one set point in the reference scale design space to one set point in the target scale design space, methods and apparatus disclosed herein permit the identification of an entire design space (e.g., space of possible set points calculated for the target scale and/or the reference scale). In some examples, a user navigates the expanded design space and identifies settings that will support a given bioprocess. As such, the identified design space gives the user flexibility to navigate inside the design space as well as permit improved investigation of process deviations.

Additionally, methods and apparatus disclosed herein allow a user to readily access results when scaling with different bioreactor configurations, as well as easily compare results according to various criteria. In some examples, methods and apparatus disclosed herein also initiate warnings to the user (e.g., low oxygen transfer, risk of carbon dioxide accumulation, etc.) based on real-time data and/or extrapolations performed during the bioreactor scaling process. Examples disclosed herein permit the identification of acceptable variable value ranges for a target bioreactor (e.g., agitation, aeration) based on a specific target bioreactor configuration, cell culture information, identification of reference bioreactor-based variable values (e.g., tip speed, mixing time, etc.) and/or the identification of set points that reduce deviations from reference bioreactor-based variable values. Additionally, method and apparatus disclosed herein introduce a simulation functionality that gives a user the ability to explore a design space and find other settings that maintain desired criteria for the bioprocess. While the examples disclosed herein focus on scaling up a process, the methods and apparatus disclosed herein can be applied to scaling down a process and/or can be used in any other applications requiring scaling of a specific process not limited to bioprocessing applications.

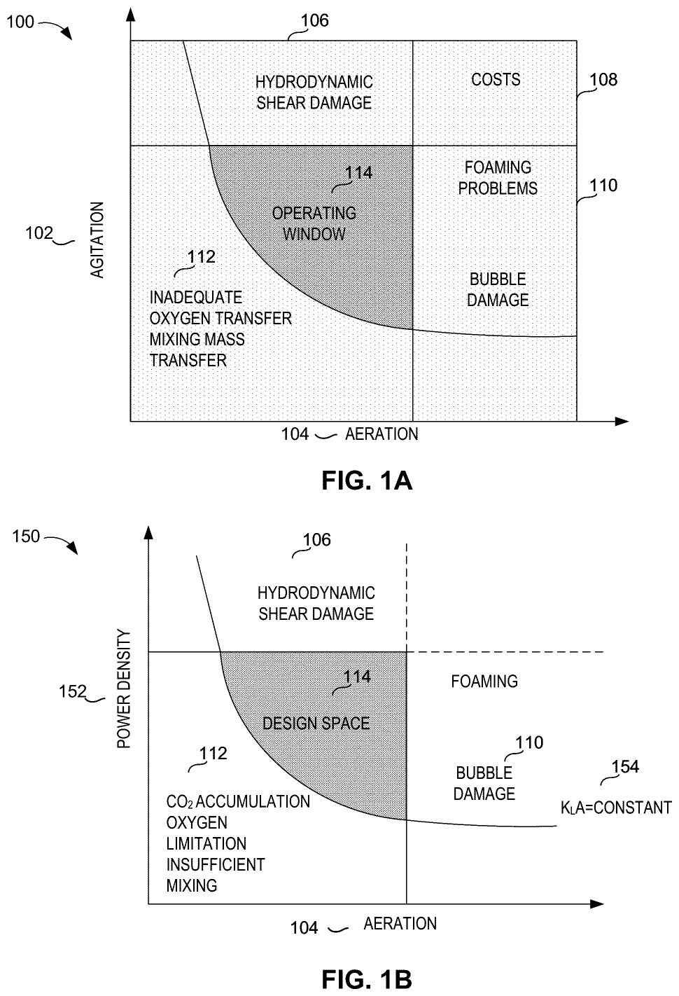

FIG. 1A illustrates an example graph 100 depicting an example operating window 114 for example aeration 104 and example agitation 102 in bioprocess systems. FIG. 1B illustrates example graph 150 showing bioreactor scale-up parameters that can be kept constant during bioreactor scaling. Bioreactor operating parameters (e.g., gas flow rate, agitation 102, etc.) can be bound by upper and lower limits. For example, oxygen transfer can be restricted when operation occurs below a lower limit and/or lower bound for oxygen flow. As such, an operating window and/or a design space 114 is an important consideration when scaling a process. For example, a design space 114 can illustrate an acceptable range for power density (P/V) 152 and aeration 104. For example, a specific range can exist for sufficient oxygen transfer and mixing that avoids the production of foaming (e.g., foaming problems and/or bubble damage 110), high carbon dioxide concentration (e.g., inadequate oxygen transfer and/or mixing 112), and/or impeller shear (e.g., hydrodynamic shear damage 106). In some examples, a design space 114 can be smaller for larger sized bioreactors when compared to smaller bioreactors due to mass transfer limitations. In some examples, bioreactor scale-up parameters that can be kept constant during scaling can include power density 152 and volumetric oxygen transfer coefficient (kLa) 154, as well as mixing time, tip speed, shear rate, and/or volumetric gas flow rates. A scaling strategy developed based on bioreactor scale-up parameters includes a comparison of two bioreactors, a reference bioreactor (e.g., initially used to develop the bioprocess) and a target bioreactor (e.g., used to create bioprocess-based products on a large scale). When scaling between two bioreactors (e.g., a reference bioreactor and a target bioreactor), process performance can be evaluated, including the resulting product quality and overall scalability (e.g., from a lab-scale to an industrial-scale). In some examples, scaling can include calculation and testing of agitation and gas settings suitable for a specific cell culture used in the bioprocess. In some examples, the agitation 102 can be selected with the intention of keeping the power density 152 constant.

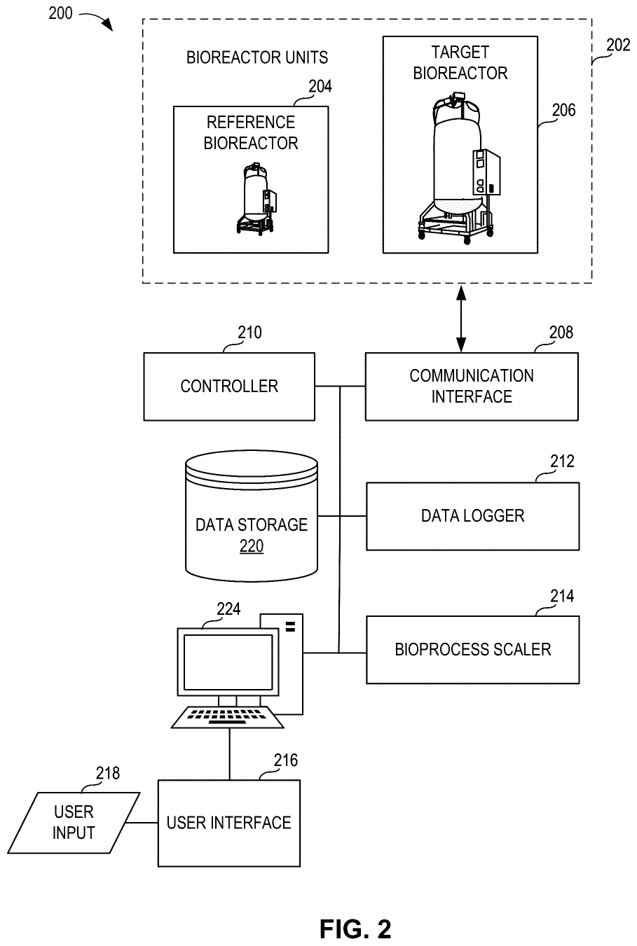

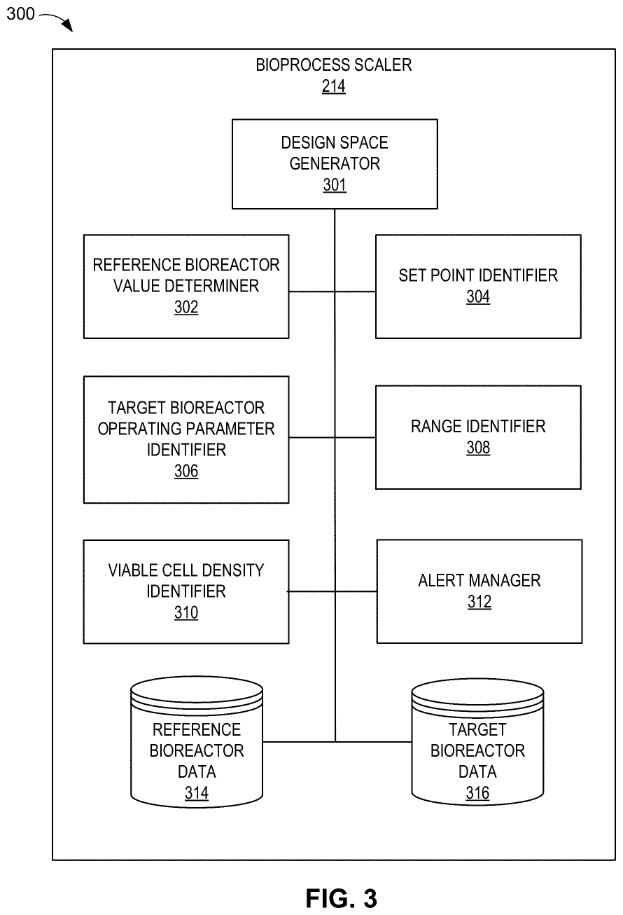

Overall, scaling should involve consideration of parameters affecting mass transfer as well as target operating parameters across all potential scales, with the operating window and/or design space 114 varying across the different scales. However, navigation between multiple scaling criteria can be challenging when attempting to remain within the design space 114. For example, while certain parameters can be kept constant (e.g., tank diameter, impeller diameter, etc.) without significant effort, other parameters are not scalable and remain constant across scales (e.g., cell size, gas bubble size, etc.). As such, navigation between different scaling criteria (e.g., energy dissipation rates, mass transfer coefficients, mixing times, etc.) is performed to achieve proper scaling and maintain the process in the intended design space 114. As described in the examples disclosed herein in connection with FIGS. 2 and 3, a scaling strategy can be implemented that allows a user improved flexibility in navigating within the design space 114 for a particular scaling from a reference bioreactor to a target bioreactor, such as exploring the design space 114 to identify other settings that maintain desired criteria for the bioprocess.

FIG. 2 is a block diagram illustrating an example environment 200 for bioreactor scaling in accordance with the teachings of this disclosure. The example environment 200 includes example bioreactor units 202, an example communication interface 208, an example controller 210, an example data logger 212, an example bioprocess scaler 214, an example user interface 216, an example user input 218, an example data storage 220, and/or an example workstation 224.

The bioreactor units 202 include an example reference bioreactor 204 and/or an example target bioreactor 206. The bioreactor units 202 can include any type of bioreactor used in a bioprocess. For example, the reference bioreactor 204 can be a microscale bioreactor, while the target bioreactor 206 can be a large-scale bioreactor (e.g., a single use bioreactor, etc.). In some examples, the bioreactors 204, 206 can include any type of commercial bioreactor (e.g., a stirred tank bioreactor, an airlift bioreactor, etc.) such as an Xcellerex bioreactor (XDR 10, XDR 50, XDR 200, XDR 500, XDR 1000, XDR 2000) and/or an Ambr® bioreactor (e.g., Ambr®15, Ambr®250, etc.). The bioreactors can be any type of unit and/or instrument used during biomanufacturing, from initial biomass expansion and media preparation to final product collection and purification. In some examples, other bioreactor units 202 can include a mixer (e.g., a jacketed mixer, a single wall mixer, etc.), a fermenter, or any other type of equipment that may be used during bioprocessing. In some examples, the bioreactors 204, 206 can be used for expansion (e.g., growth of CHO cells, bacteria, yeast, etc.) to permit biological reactions under controlled conditions for a variety of purposes, including the production of pharmaceuticals, vaccines, antibodies, and biofuel. Such bioreactors can be used in any domain of industrial biotechnology requiring large scale production, providing the necessary biological, biochemical, and biomechanical conditions for synthesis of desired products.

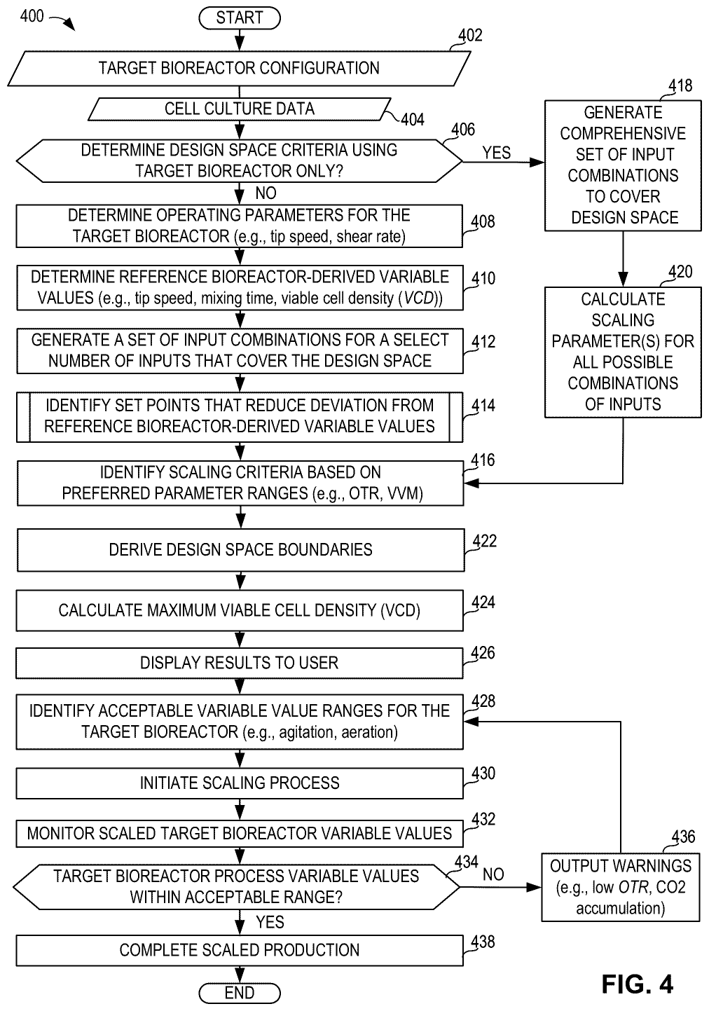

The communication interface 208 can communicate with the bioreactor units 202 (e.g., reference bioreactor 204, target bioreactor 206) via wired and/or wireless-based Ethernet. In some examples, the communication interface 206 permits identification of bioreactor 204, 206 locations. In some examples, the communication interface 208 is used to determine bioreactor settings (e.g., configurations) based on receipt of information from the bioreactor units 202. For example, the communication interface 208 can be used to receive bioreactor 204, 206 settings and/or modify bioreactor 204, 206 settings based on input from the controller 210 and/or user-based settings and/or inputs (e.g., via the user interface 216). In some examples, the communication interface 208 can be used to receive data from the bioreactor 204, 206 as part of tracking bioreactor 204, 206 performance and/or identifying bioreactor 204, 206 operating parameters. In some examples, the communication interface 208 can be used to receive data needed for determining reference bioreactor 204 variable values (e.g., tip speed, mixing time, viable cell density, etc.), data needed for determining operating parameters for the target bioreactor 206 (e.g., top speed, shear rate, etc.), and/or data needed to determine acceptable variable value ranges for the target bioreactor 206 (e.g., agitation, aeration), as described in connection with FIGS. 4 and/or 5. In addition, the communication interface 208 can be used to monitor scaled target bioreactor 206 variable values during the bioprocess and/or identify potential deviations from the desired design space 114 of FIG. 1.

The controller 210 (e.g., a programmable logic controller) is in communication with the bioreactor units 202 via the communication interface 208. In some examples, the controller 210 evaluates whether the bioreactor is maintaining a proper controlled environment for biomass expansion (e.g., temperature, pH, oxygen, carbon dioxide, etc.). In some examples, the controller 210 evaluates critical parameters (e.g., logged via the data logger 212) to determine whether they are within an acceptable range and/or require adjustment. In some examples, the controller 210 monitors and/or optimizes various conditions and/or parameters that affect the results of the bioreactor-based scale-up (e.g., gas distribution, mixing time, heat-transfer rate, mass-transfer coefficients, volumetric power input, etc.).

The data logger 212 logs data associated with the bioprocess performed using the bioreactor units 202. For example, the data logger 212 can log real-time pH readings and/or osmolarity readings (e.g., using near-infrared spectroscopy, optical sensors, etc.). In some examples, the data logger 212 can be used to identify information related to bioreactor 204, 206 cell culture data, bioreactor 204, 206 configuration, and/or any other parameters that can be identified via a direct and/or indirect measurement (e.g., a sensor-based measurement of carbon dioxide and/or oxygen levels, etc.). In some examples, the data logger 212 can be used to compare the real-time settings of the bioreactors 204, 206 to desired settings input by the user (e.g., via user input 218).

The bioprocess scaler 214 permits the scaling from a reference bioreactor 204 to a target bioreactor 206. In some examples, the bioprocess scaler 214 can include a reference bioreactor value determiner, a set point identifier, a target bioreactor operating parameter identifier, a range identifier, and/or a viable cell density identifier. The bioprocess scaler 214 can be used to identify values associated with parameters that affect bioreactor scaling (e.g., tip speed, energy dissipation rate, mixing time, mass transfer coefficient, turbulent shear force, etc.). For example, the bioprocess scaler 214 can be used to stay in the same operating window 114 of FIG. 1 across all scales. In some examples, the bioprocess scaler 214 can identify anticipated changes in gas flow over time based on the bioreactor sparger type and/or overall mixing speed and aeration. In some examples, the bioprocess scaler 214 can determine the sparger type (e.g., 2 micrometer sparger, 20 micrometer sparger, 1 millimeter sparger, etc.) that meets the carbon dioxide removal requirement and/or achieves a desired volumetric oxygen transfer coefficient (kLa) value. In some examples, the bioprocess scaler 214 identifies reference bioreactor 204 variable values (e.g., tip speed, mixing time, etc.) and calculates the optimal operating parameters for the target bioreactor 206 (e.g., mixing speed, gas flow rates, etc.), as described in connection with FIG. 4. In some examples, the bioprocess scaler 214 computes mixing time, power input, and/or the volumetric oxygen transfer coefficient (kLa) for any given set point. In some examples, the bioprocess scaler 214 can generate a kLa versus sparging rate plot. The bioprocess scaler 214 can be used to perform scaling for various bioreactor configurations, taking into consideration fulfillment of requirements associated with oxygen transfer and/or carbon dioxide stripping, resulting in the identification of set points (e.g., agitation speed, sparger configuration, air flow, oxygen flow, etc.) based on the desired set of requirements, allowing comparison of results based on different criteria (e.g., as specified by a user via user input 218). In some examples, the bioprocess scaler 214 produces warnings to the user indicating reduced oxygen transfer or increased risk of carbon dioxide accumulation. Additionally, the bioprocess scaler 214 permits user navigation within the design space 114 for a particular scaling from the reference bioreactor 204 to the target bioreactor 206, including the exploration of the design space 114 to identify other settings that maintain desired criteria for the bioprocess. While in the example of FIG. 2 the bioprocess scaler 214 is integrated into the bioprocess system, the bioprocess scaler 214 can be a stand-alone software program that can be used separately from the bioprocess system to model and/or determine optimal bioreactor operating parameters during scaling. As such, a user can input data into the bioprocess scaler 214 to perform scaling from the reference bioreactor 204 to the target bioreactor 206 without the scaler 214 being in communication with the bioreactor units 202.

The user interface 216 is in communication with the workstation 224, allowing user-based input(s) 218 that can provide preferred bioreactor 204, 206 settings and/or operating parameters. In some examples, the user interface 216 can be used to display data associated with scaling from the reference bioreactor 204 to the target bioreactor 206 (e.g., plots, operating parameter values, target values, etc.). In some examples, the user interface 216 can be used to display warnings associated with the bioprocess (e.g., reduced oxygen transfer, increased risk of carbon dioxide accumulation, etc.). In some examples, the user interface 216 presents a real-time overview of the bioreactor operating parameters and allows a user to make any necessary and/or desired modifications. For example, the user interface 216 can present reference and/or target bioreactor data information to the user. Likewise, the user interface 216 can be used for navigation of the software associated with the bioprocess scaler 214. In some examples, the user interface 216 changes based on user-provided selections, including the type of information available to the user. For example, the user interface 216 can present different selections and/or options if the user has liquid mixing model information versus if the user has physical characterization data available but may not have any modeling experience.

The data storage 220 stores any logged data and/or any other information received from the bioreactor units 202 during operation. In some examples, the data storage 220 includes data related to bioprocess unit 204, 206 location, status, mode and/or other information related to bioprocess unit 204, 206 usage (e.g., maintenance, configuration, battery status, upstream/downstream processing tasks, etc.). The data storage 220 can be implemented by any storage device and/or storage disc for storing data such as, for example, flash memory, magnetic media, optical media, web-based storage, private cloud storage, etc. Furthermore, the data stored in the data storage 220 can be in any data format such as binary data, comma delimited data, tab delimited data, structured query language (SQL) structures, etc. While in the illustrated example the data storage 220 is illustrated as a single database, the data storage 220 can be implemented by any number and/or type(s) of databases.

The workstation 224 permits a user to operate and/or monitor the bioprocess units 202. The workstation 224 is communicatively coupled to the controller 210, the communication interface 208, the data logger 212, and/or the data storage 220 via a bus or local area network (LAN) (e.g., an Area Control Network (ACN)). The LAN can be implemented using any desired communication medium and protocol. For example, the LAN can be based on a hardware or wireless Ethernet communication protocol. However, any other suitable wired or wireless communication medium and protocol could be used. The workstation 224 can be configured to perform operations associated with one or more information technology applications, user-interactive applications (e.g., via the user interface 216), and/or communication applications. For example, the workstation 224 can be configured to perform operations associated with process control-related applications and communication applications that enable the workstation 224 and the controller 210 to communicate with other devices or systems using any desired communication media (e.g., wireless, hardwired, etc.) and protocols (e.g., HTTP, SOAP, etc.).

FIG. 3 is a block diagram of an example implementation 300 of the bioprocess scaler 214 of FIG. 2 to facilitate bioprocess instrument scaling. The bioprocess scaler 214 includes an example design space generator 301, an example reference bioreactor value determiner 302, an example set point identifier 304, an example target bioreactor operating parameter identifier 306, an example range identifier 308, an example viable cell density identifier 310, an example alert manager 312, an example reference bioreactor data storage 314, and/or an example target bioreactor data storage 316.

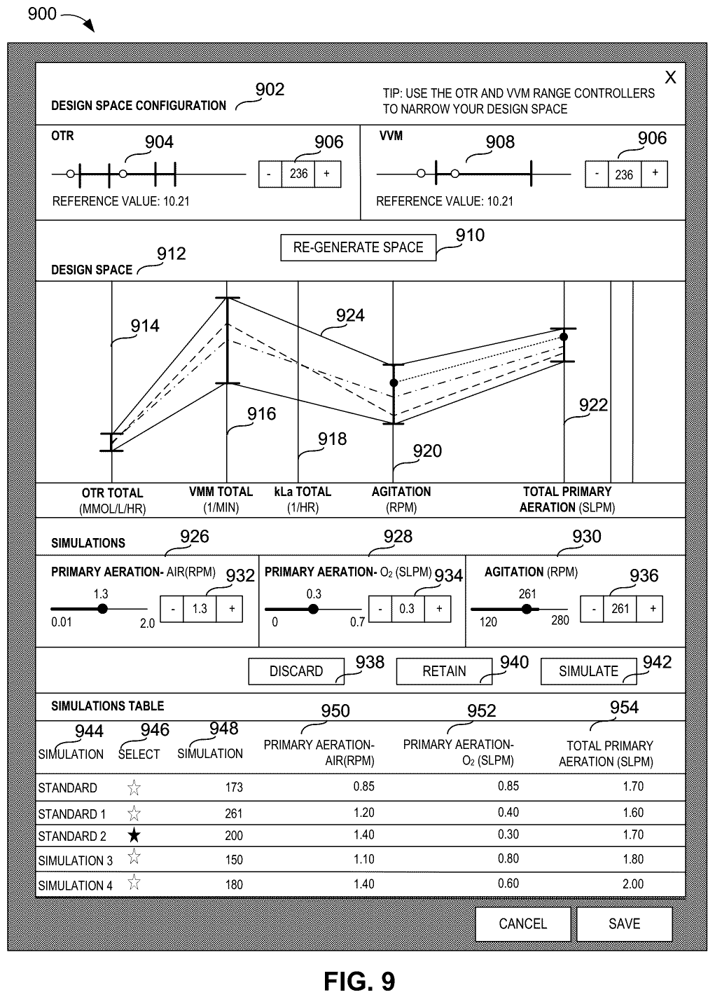

The design space generator 301 determines design space criteria for performing the bioprocess scaling. In some examples, the design space generator 301 determines design space criteria based on whether a target scale is used or whether a combination of the target scale and reference scale is used to identify scaling criteria. In some examples, the design space generator 301 identifies operating parameters for the target bioreactor (e.g., tip speed, shear rate, etc.) and/or reference bioreactor-derived variable values(s) (e.g., tip speed, mixing time, viable cell density (VCD)). In some examples, the design space generator 301 generates a comprehensive set of input combinations to cover a given design space. In some examples, the design space generator 301 generates a set of input combinations for a select number of inputs that cover the design space. Once the set of inputs is determined, the design space generator 301 can identify set points that reduce deviations from the reference bioreactor-derived variable values, thereby identifying scaling criteria based on preferred parameter ranges (e.g., OTR, VVM). However, in some examples, the design space generator 301 can also calculate scaling parameter(s) for all possible combinations of inputs associated with a comprehensive set of inputs to cover the design space. Once scaling criteria are identified, the design space generator 301 can likewise be used to derive the design space boundaries, as described in connection with FIG. 9.

The reference bioreactor value determiner 302 determines reference bioreactor-derived variable values. For example, the reference bioreactor value determiner 302 calculates power consumption per liquid volume (P/V), tip speed, mixing time, shear rate, primary sparger values (e.g., kLa, oxygen transfer rate, ratio of total aeration to the bioreactor working volume (VVM), oxygen transfer rate (OTR), etc.), and/or secondary sparger values (e.g., kLa, oxygen transfer rate, ratio of total aeration to the bioreactor working volume (VVM), oxygen transfer rate (OTR), etc.). In some examples, the reference bioreactor value determiner 302 calculates viable cell density (VCD) (e.g., a maximum VCD value) based on the primary sparger and/or secondary sparger values). In some examples, such calculations are based on input received from the bioreactors 204, 206 and/or user-based input 218 of FIG. 2. For example, input can include bioreactor-based configuration data, existing bioreactor and/or scaling model data, and/or cell line-based data.

The set point identifier 304 determines set points that reduce deviations from reference bioreactor-derived variable values. In some examples, the set point identifier 304 determines a set point based on user-based input 217 (e.g., revolutions per minute). Based on user-provided input, the set point identifier 304 calculates target bioreactor-based operation parameters (e.g., P/V, tip speed, mixing time, shear rate, etc.). In some examples, the set point identifier 304 identifies the set point based on data from the primary and/or secondary sparger. For example, a minimum and a maximum oxygen transfer rate (OTR) can be determined based on the target bioreactor operating parameter calculations. In some examples, the set point identifier 304 determines secondary and/or primary aeration based on the primary and/or secondary spargers in use by a target bioreactor (e.g., target bioreactor 206). The set point identifier 304 reduces percentage differences from the reference bioreactors to the target bioreactor values, while varying the primary and/or secondary aeration within valid range(s) using the range identifier 308. In some examples, the set point identifier 304 determines whether the process proceeds via a default method of determining set points or via a simulation. For example, a user can select to run a simulation to explore a design space and identity other settings that maintain desired criteria. For example, in a simulation, the target revolutions per minute can be set by the user, rather than being calculated for matching a reference P/V within a test range. In some examples, a Newton (TNC) algorithm can be used to solve for an RPM for a given P/V.

The target bioreactor operating parameter identifier 306 determines target bioreactor derived variables. For example, the target bioreactor operating parameter identifier 306 calculates P/V, tip speed, mixing time, shear rate, primary sparger kLa, primary sparger VVM, and/or primary sparger OTR. In some examples, the target bioreactor operating parameter identifier 306 determines secondary sparger kLa, VVM and/or OTR if the target bioreactor 206 includes a secondary sparger. The target bioreactor operating parameter identifier 306 can be used to calculate a maximum viable cell density (VCD) as part of determining the target variables for the target bioreactor 206.

The range identifier 308 determines a range of values that can be used for a given target bioreactor 206 setting and/or configuration while still allowing the values to stay within an acceptable range that correspond to an identified design space 114 of FIG. 1. For example, the range identifier 308 can determine a specific characterization range to be used for revolutions per minute (RPM) which can vary depending on the type of bioreactor being used and/or bioreactor configuration. In some examples, the characterization range can include a specific range of revolutions per minute for a given volume (e.g., 40-360 RPM per 4.5-10 L). In some examples, the user interface 216 can include a slider that indicates the range of values for which a bioreactor's physical characterization data exists.

The viable cell density identifier 310 determines a maximum viable cell density (VCD) and/or viable cell concentration (VCC). For example, the viable cell density determiner 310 can identify the maximum VCD (e.g., in million cells per milliliter) that can be supported by the oxygen transfer rate (OTR) achieved. In some examples, the viable cell density identifier 310 can use the calculated OTR to derive the cell specific oxygen consumption rate (qO₂) for a given cell line at a given time.

The alert manager 312 identifies any deviations in the process from the intended operating values and triggers an alert to notify the user. For example, the alert manager 312 can be used to produces warnings to the user indicating reduced oxygen transfer or increased risk of carbon dioxide accumulation. In some examples, the alert manager 312 tracks user-provided inputs and identifies any deviations outside of the design space 114 of FIG. 1 that could result in reduced product quality and/or production. In some examples, the alert manager 312 tracks any deviations in operating parameters to identify potential areas for correction (e.g., mixing speed, etc.). For example, the alert manager 312 can provide the user with warnings and/or alerts when target bioreactor process variable values are outside an acceptable range as determined using the range identifier 308. In some example, automatic corrections can be made instead of requiring user intervention to adjust bioreactor operating parameters.

The reference bioreactor data storage 314 stores any data associated with the reference bioreactor (e.g., bioreactor 204 of FIG. 2). In some examples, the data storage 314 stores any data input by a user that can be used to determine reference bioreactor parameters. The data storage 314 can be implemented by any storage device and/or storage disc for storing data such as, for example, flash memory, magnetic media, optical media, web-based storage, private cloud storage, etc. Furthermore, the data stored in the data storage 314 can be in any data format such as, for example, binary data, comma delimited data, tab delimited data, structured query language (SQL) structures, etc. While in the illustrated example the data storage 314 is illustrated as a single database, the data storage 314 can be implemented by any number and/or type(s) of databases.

The target bioreactor data storage 316 stores any data associated with the target bioreactor (e.g., bioreactor 206 of FIG. 2). In some examples, the data storage 316 stores any data input by a user that can be used to determine target bioreactor parameters. The data storage 316 can be implemented by any storage device and/or storage disc for storing data such as, for example, flash memory, magnetic media, optical media, web-based storage, private cloud storage, etc. Furthermore, the data stored in the data storage 316 can be in any data format such as, for example, binary data, comma delimited data, tab delimited data, structured query language (SQL) structures, etc. While in the illustrated example the data storage 316 is illustrated as a single database, the data storage 314 can be implemented by any number and/or type(s) of databases.

While an example implementation of the bioprocess scaler 214 is illustrated in FIG. 3, one or more of the elements, processes and/or devices illustrated in FIG. 3 may be combined, divided, re-arranged, omitted, eliminated and/or implemented in any other way. Further, the example design space generator 301, the example bioreactor value determiner 302, the example set point identifier 304, the example target bioreactor operating parameter identifier 306, the example range identifier 308, the example viable cell identifier 310, the example alert manager 312, and/or, more generally, the example bioprocess scaler 214 of FIG. 3 may be implemented by hardware, software, firmware and/or any combination of hardware, software and/or firmware. Thus, for example, any of the example design space generator 301, the example bioreactor value determiner 302, the example set point identifier 304, the example target bioreactor operating parameter identifier 306, the example range identifier 308, the example viable cell identifier 310, the example alert manager 312, and/or, more generally, the example bioprocess scaler 214 of FIG. 3 can be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example design space generator 301, the example bioreactor value determiner 302, the example set point identifier 304, the example target bioreactor operating parameter identifier 306, the example range identifier 308, the example viable cell identifier 310, the example alert manager 312, and/or, more generally, the example bioprocess scaler 214 of FIG. 3 is/are hereby expressly defined to include a non-transitory computer readable storage device or storage disk such as a memory, a digital versatile disk (DVD), a compact disk (CD), a Blu-ray disk, etc. including the software and/or firmware. Further still, the example bioprocess scaler of FIG. 3 may include one or more elements, processes and/or devices in addition to, or instead of, those illustrated in FIG. 3, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

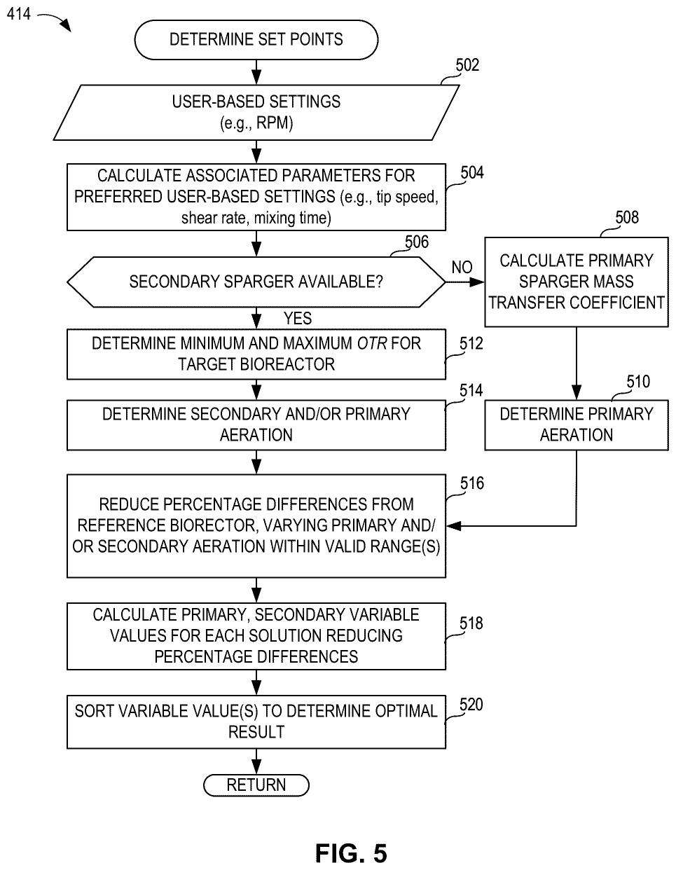

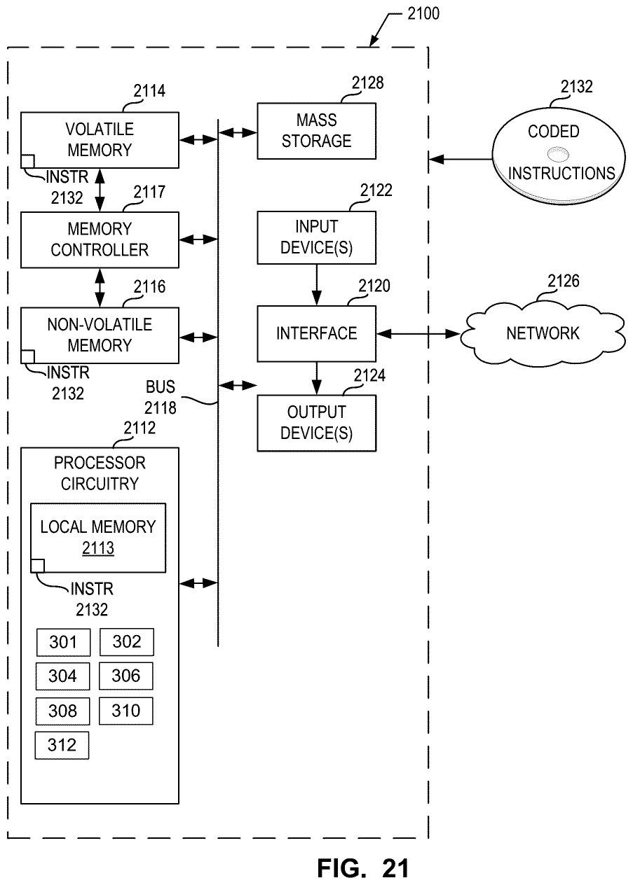

Flowcharts representative of example hardware logic, machine readable instructions, hardware implemented state machines, and/or any combination thereof for implementing the bioprocess scaler 214 of FIG. 3 are shown in FIGS. 4-5. The machine readable instructions may be one or more executable programs or portion(s) of an executable program for execution by a computer processor such as the processor 2112 shown in the example processor platform 2100 discussed below in connection with FIG. 21. The program may be embodied in software stored on a non-transitory computer readable storage medium such as a CD-ROM, a floppy disk, a hard drive, a DVD, a Blu-ray disk, or a memory associated with the processor 2112, but the entire program and/or parts thereof could alternatively be executed by a device other than the processor 2112 and/or embodied in firmware or dedicated hardware. Further, although the example program is described with reference to the flowchart illustrated in FIGS. 4-5, many other methods of implementing the example bioprocess scaler 214 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, Ladder Logic, Function Block Diagram (FBD), Structured Text, Sequential Flow Charts, Instruction List, etc.

As mentioned above, the example processes of FIGS. 4-5 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

FIG. 4 is a flowchart representative of example machine-readable instructions 400 that may be executed during bioreactor scaling. In the example of FIG. 4, the bioprocess scaler 214 receives inputs such as target bioreactor 206 configuration (block 402). In some examples, a user can provide the target bioreactor configuration information via the user input 218 to the user interface 216 and/or the bioreactor configuration can be obtained from the bioreactor via the communication interface 208. Additional inputs to the bioprocess scaler 214 can include cell culture data (block 404). In some examples, a user can select a pre-existing cell line and its corresponding information will be used in the scaling calculation. In some examples, a cell line that is biologically close to any other known cell line can be used. In some examples, a user can add specific information regarding a new cell line. Once the bioreactor configuration and cell culture data are provided, the design space generator 301 determines design space criteria based on whether a target scale is used alone or in combination with a reference scale to obtain desired scaling of the bioreactor (block 406). If the design space generator 301 determines that both the target bioreactor and the reference bioreactor are to be considered in the scaling process, the target bioreactor operating parameter identifier 306 determines operating parameters for the target bioreactor (block 408) while the reference bioreactor value determiner 302 determines reference bioreactor-derived variable values (block 410). For example, as described in connection with FIG. 3, the reference bioreactor value determiner 302 can calculate values specific to the reference bioreactor, such that these values can be used when identifying set points that reduce any deviations from the reference bioreactor to the target bioreactor. For example, the reference bioreactor value determiner 302 can calculate power consumption per liquid volume (P/V), tip speed, mixing time, shear rate, primary sparger values, and/or secondary sparger values. The design space generator 301 can proceed to generate a set of input combinations for a select number of inputs that cover the design space (block 412). The set of input combinations can be further used by the set point determiner 304 to determine the set points that reduce deviation from reference bioreactor-derived variable values (block 414). As described in more detail in connection with FIG. 5, the set point determiner 304 calculates bioreactor parameters based on user-based settings. Once the set points have been identified, the design space generator 301 can identify scaling criteria based on preferred parameter ranges (e.g., OTR, VVM ranges) (block 416). As such, the design space generator 301 can be used to derive design space boundaries (block 422).

In some examples, the design space generator 301 generates a comprehensive set of input combinations to cover the design space (block 418). For example, when the design space generator 301 determines that design space criteria are to be identified based on the target bioreactor only, the set of potential input combinations to explore as part of the potential design space increases. As such, the design space generator 301 calculates scaling parameter(s) for all possible combinations of inputs (block 420), thereby not limiting the combinations of inputs to a select number of inputs (e.g., as described in connection with block 412). Once the scaling parameter(s) have been calculated as part of block 420, control proceeds to block 416, allowing the design space generator 301 to identify scaling criteria based on the preferred parameter ranges (e.g., OTR, VVM) and/or derive the design space boundaries (block 422). Additionally, the viable cell density identifier 310 can be used to calculate a maximum viable cell density (VCD) based on the identified variable values sorted using the set point identifier 304 (block 424). Results of the scaling can be presented to the user using the user interface 216 of FIG. 2 (block 426). In some examples, the range identifier 308 determines acceptable variable value range(s) for the target bioreactor (block 428). For example, the bioprocess scaler 214 can suggest agitation and aeration values to match OTR. In some examples, various simulations can be performed to identify the agitation and aeration based on specific criteria. In some examples, the bioprocess scaler 214 presents the user, via the user interface 216, with an output including bioreactors used for the target scale and/or the reference scale, with a comparison of the settings and/or values determined for the reference and target bioreactors, as well as data associated with the primary and/or secondary spargers. Such an output can include graphical representations of the data and/or comparisons between the reference scale, the target scale, and/or results of simulations performed based on user-specified values.

In some examples, such as when the bioprocess scaler 214 is integrated into a given bioreactor system, the bioprocess scaler 214 initiates the scaling process (block 430). For example, the scaling initiation can result in the target bioreactor 206 engagement to initiate a given bioprocess based on the determined target bioreactor operating parameters and/or acceptable value ranges for the target bioreactor that permits the scaling process to remain within a given design space 114 of FIG. 1. Once the scaling process is initiated, the bioprocess scaler 214 and/or the controller 210 monitors the process (block 432). For example, the bioprocess scaler 214 can be used to identify any deviations of the target bioreactor process variable values from the acceptable range(s) (block 434). If deviations are observed, the alert manager 312 can output, via the user interface 216, a warning providing information about a given deviation (block 436). The user can interfere to make adjustments and/or the bioprocess scaler 214 reverts back to identifying acceptable variable value ranges to confirm the deviation (block 428). The controller 210 can be used to initiate completion of the scaled production using the target bioreactor 206 if the target bioreactor-based process does not initiate further warnings to the user (block 438).

FIG. 5 is a flowchart representative of example machine-readable instructions 414 that may be executed to determine set points during bioreactor scaling. In the example of FIG. 5, the set point identifier 304 receives user-based settings to use these settings to set a target point (block 502). In some examples, the set point identifier 304 calculates associated parameters for preferred user-based settings (block 504). If a secondary sparger is available (block 506), the set point identifier 304 determines minimum and maximum oxygen transfer rate values for the target bioreactor 206 (block 512) and calculates primary and/or secondary aeration based on the sparger type (block 514). If a secondary sparger is not available, the set point identifier 304 calculates the primary sparger-based mass transfer coefficient (block 508). As such, the primary aeration can be determined based on the mass transfer coefficient calculation (block 510). Once relevant values are determined for a bioreactor with a primary sparger and/or a bioreactor with a primary and a secondary sparger, the set point identifier 304 reduces percentage differences between the target and reference bioreactors, varying the primary and/or secondary aeration with valid range(s) (block 516). For example, the set point identifier 304 determines an upper and a lower bound for the primary and/or secondary aeration values, allowing the design space 114 of FIG. 1 to be maintained. The set point identifier 304 further calculates primary and/or secondary variable values, reducing percentage differences (bock 518). For example, the set point identifier 304 reduces the OTR difference, the VVM difference, and/or the kLa difference between the reference and target bioreactors. For various solutions that are obtained by reducing these differences, the set point identifier 304 sorts the solution based on, for example, ascending OTR difference, ascending VVM difference, ascending kLa difference, and/or descending primary aeration (block 520).

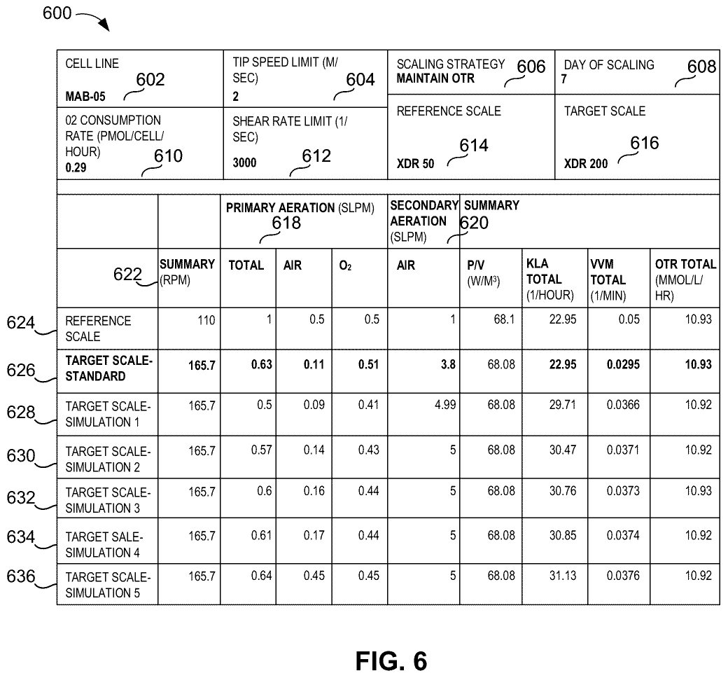

FIG. 6 illustrates an example user interface 600 including a target bioreactor and a reference bioreactor and their associated target and reference scales, respectively. In the example of FIG. 6, the user interface 600 includes a display showing an example cell line 602, and example tip speed limit 604, an example scaling strategy 606 (e.g., maintaining the oxygen transfer rate), an example day of scaling 608 (e.g., day 7), an example oxygen consumption rate 610, an example shear rate limit 612, an example reference scale 614, and an example target scale 616. In some examples, the user interface can include values for an example primary aeration 618, an example secondary aeration 620, and an example parameter identification 622 (e.g., agitation, P/V, kLa, etc.). In addition, the user interface 600 includes values determined for the reference scale 624, the target scale 626, and example simulation(s) 628, 630, 632, 634, 636.

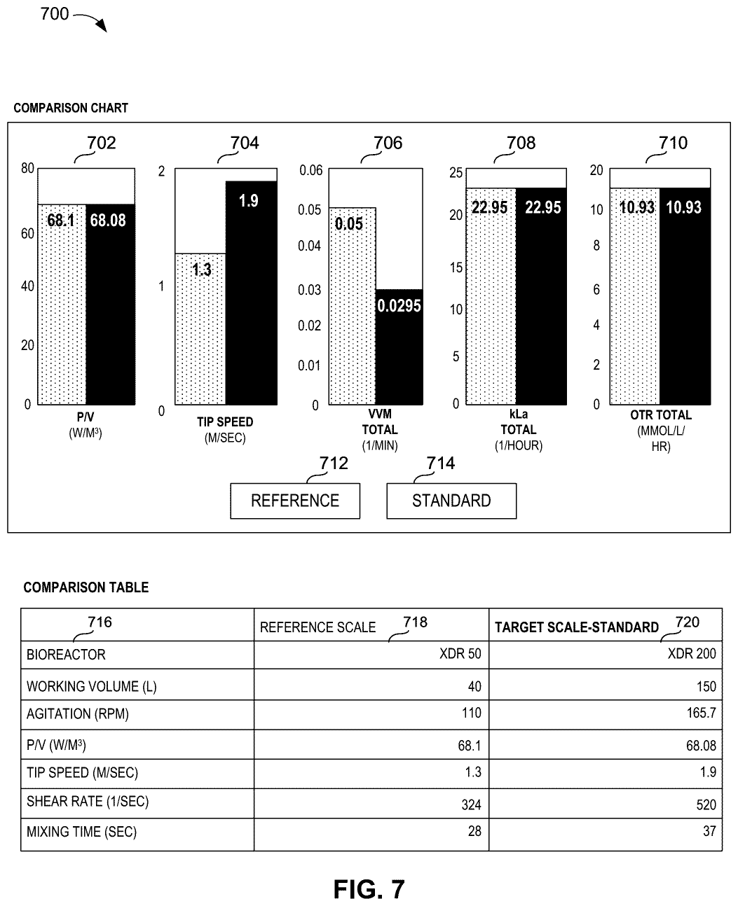

FIG. 7 illustrates an example user interface 700 including reference and standard values for multiple variables during bioreactor scaling. In the example of FIG. 7, a range of parameters are identified to show their comparison between the reference scale 712 and the standard scale 714 (e.g., target scale). The parameters can include example P/V 702, example tip speed 704, example VVM total 706, example kLa total 708, and/or example OTR total 710. Additionally, the user interface 700 includes a comparison table displaying the listed values for the reference scale 718 and the target scale 720 for operating parameters 716 (e.g., agitation, mixing time, etc.). In the example of FIG. 7, a simulation is not in progress and/or has not been performed, thereby a separate bar graph for simulation results of simulations 1-5 shown in FIG. 6 are not included.

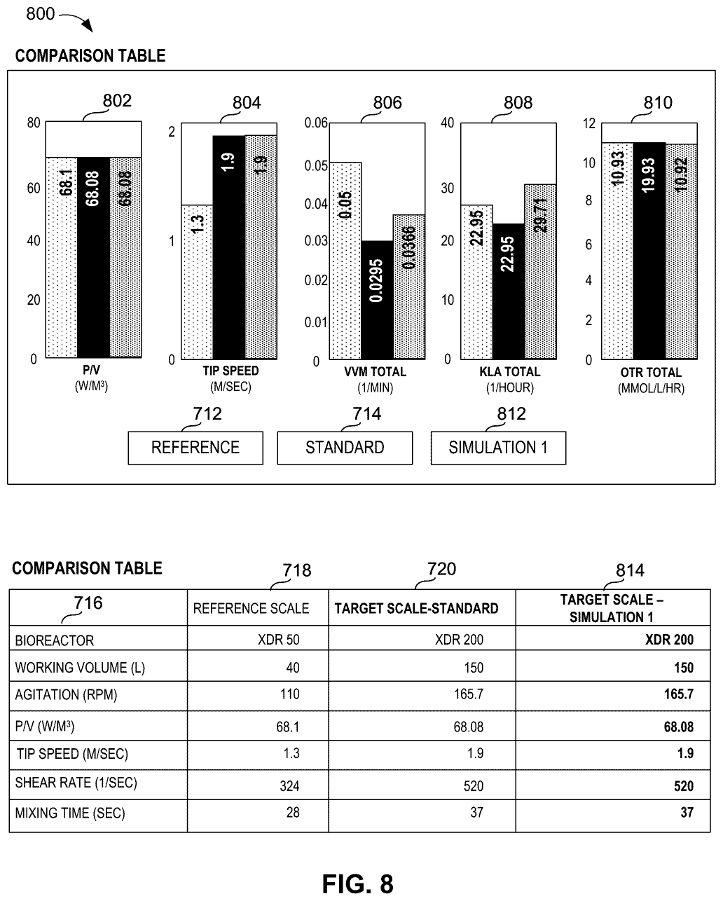

FIG. 8 illustrates an example user interface 800 including an example simulation functionality that gives a user the ability to explore a design space and find other settings that maintain desired criteria. In the example of FIG. 8, simulation values are displayed alongside the reference and/or standard values. For example, values shown for example P/V calculation 802, example tip speed calculation 804, example VVM total calculation 806, example kLa total calculation 808, and/or example OTR total calculation 810 are displayed for the reference scale 712, the standard scale 714, and/or a specific simulation 812 (e.g., simulation #1). The example user interface 800 of FIG. 8 further includes a listing of the values for the simulation results 814, in addition to the values obtained by default using the reference and/or target scales. In some examples, the simulations allow users increased flexibility in evaluating the design space 114 of FIG. 1, such that various inputs can be tested by the user to determine whether they remain the design space and/or how much deviation can be allowed before the design space 114 is exited. For example, the user can identify potential variations in the agitation and/or aeration settings by selecting a value on the upper boundaries and/or the lower boundaries to determine how the variations can influence the final bioprocess results.

FIG. 9 illustrates an example design space configuration interface 900. In the example of FIG. 9, the design space generator 301 of FIG. 3 is used to determine an example design space configuration 902 to allow the user to adjust the design space. For example, the design space configuration interface 900 of FIG. 9 can be used to visualize the individual and/or combined contribution of various bioprocess parameters. In the example of FIG. 9, the design space configuration 902 includes an example oxygen transfer rate (OTR) range controller display 904 and an example bioreactor working volume (e.g., volume of air under standard conditions per volume of liquid per minute, VVM) range controller display 908, which can be adjusted using an example range adjusted 906. Once the range of given parameters (e.g., OTR, VVM, etc.) has been selected (e.g., narrowed), a user can regenerate the design space using the example selection 910. In the example of FIG. 9, an example design space visualization 912 can be used to view a graphical representation of the parameter settings, including an example OTR total setting 914, an example VVM total setting 916, an example kLa total setting 918, an example agitation setting 920, and/or an example total primary aeration setting 922. The design space visualization 912 of FIG. 9 can thereby be used to guide a user to identify a design space in which desired process parameters can be obtained (e.g., identification of an exact boundary of the design space). In some examples, various statistical methods can be used to describe and/or evaluate the design space (e.g., full factorial, Latin hypercube, space filling, etc.). As described in connection with FIG. 4, the design space generator 301 can be used to derive design space boundaries that meet certain criteria, as identified by the user. In some examples, the design space generator 301 relies on a large set of input combinations to cover the entire design space evenly. In some examples, the design space generator 301 relies on a set of combinations for a limited number of inputs that cover the entire design space evenly. The selection between different computational models for use by the design space generator 301 of FIG. 3 can depend on required computational speed and/or available storage space. In some examples, a user can select whether to determine the design space based on both reference scales and target scales and/or the target scale only. Additionally, the design space visualization 912 can be used to observe trends in the data in a graphical format, as well as make corrections and/or necessary adjustments. Furthermore, the design space configuration 902 includes example simulations (e.g., example primary aeration (air) simulation 926, example primary aeration (oxygen) simulation 928, example agitation simulation 935, etc.) that allow for an example primary aeration (air) adjustment 932, an example primary aeration (oxygen) adjustment 934, and/or an example agitation adjustment 936. In some examples, the design space configuration 902 includes an example discard selection 938 to allow a user to discard selected data, an example retain selection 940 that allows a user to retain selected data, and/or an example simulate selection 942 that allows a user to perform a simulation. A simulations table can be included to display an example simulation type 944, an example simulation selection 946, an example primary aeration (air) value 948, an example primary aeration (oxygen) value 952, and/or an example total primary aeration value 954.

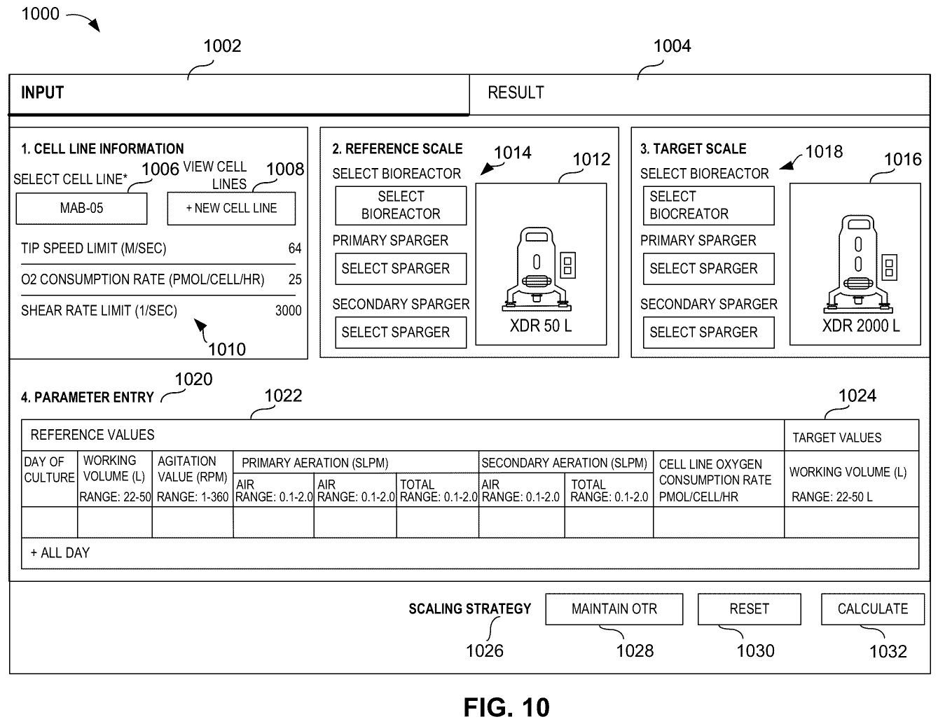

FIG. 10 illustrates an example input section 1000 indicating cell line information, reference scale information, target scale information, and parameter entry. The example input section 1000 can include an example input section tab 1002 and/or an example result section tab 1004. For example, a user can view and/or adjust cell line information, including an example cell line type 1006. Additionally, the user can view and/or add additional cell lines using an example new cell line selection 1008. The cell line information can also include example cell line identifiers 1010 (e.g., tip speed limit, oxygen consumption rate, shear rate limit, etc.) that identifies specific values that can be used for a given cell line (e.g., based on cell line type and/or sensitivity). The input section 1000 can also include example information associated with a visual representation of an example reference bioreactor view 1012 and/or an example target bioreactor view 1016. For example, reference scale information 1014 (e.g., reference bioreactor selection, primary sparger selection, secondary sparger selection, etc.) and/or example target scale information 1018 (e.g., target bioreactor selection, primary sparger selection, secondary sparger selection, etc.) can be viewed in connection with the reference bioreactor view 1012 and the target bioreactor view 1016, respectively. Furthermore, example parameter entry 1020 can be visualized using the input section 1000. The parameter entry 1020 can include the input of example reference values 1022 and/or example target values 1024 for one or more days. In some examples, the user can add additional days as needed based on a desired protocol. In some examples, the input section 1000 can include an example scaling strategy 1026. For example, the scaling strategy in FIG. 10 can be displayed in the scaling strategy display area 1028 (e.g., maintain OTR, etc.). If a user desires to reset the scaling strategy in the scaling strategy display area 1028, an example reset selection 1030 can be used to reset the scaling strategy. Once the scaling strategy has been identified, the user can proceed with the calculation(s) via an example calculate selection 1032.

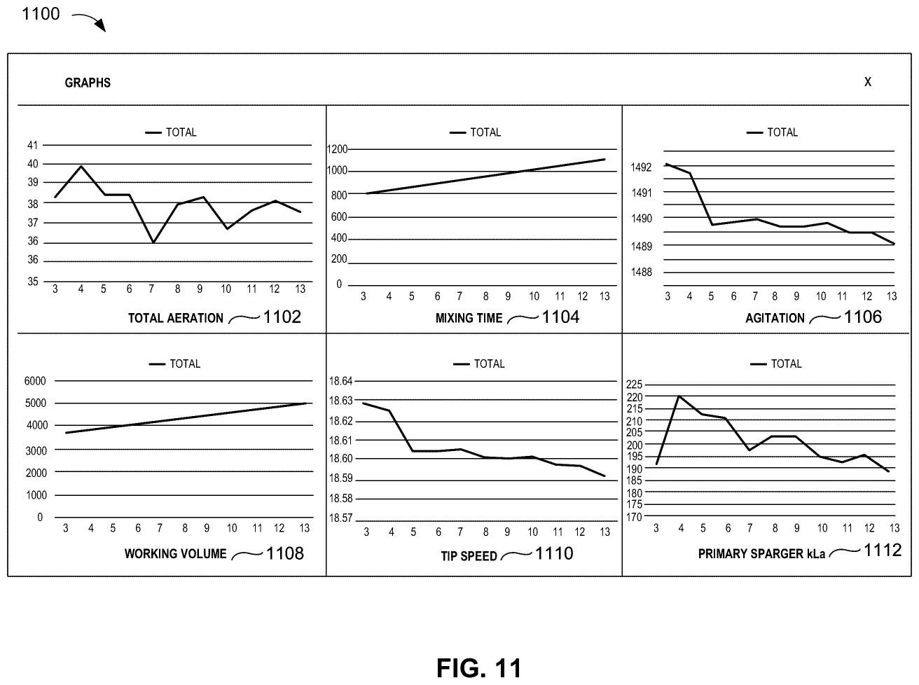

FIG. 11 illustrates an example graphical output 1100 showing results associated with variations in example aeration 1102, example mixing time 1104, example agitation 1106, example working volume 1108, example tip speed 1110, and/or example primary sparger volumetric mass transfer coefficient (kLa) 1112. For example, a user can directly visualize the output data to better evaluate ongoing trends and understand the reference process as part of scale conversion. In some examples, the user can perform multi-day parameter entry and select an option to visualize the results in a graphical format, as shown in the example of FIG. 11. As such, data for the aeration 1102, mixing time 1104, agitation 1106, working volume 1108, tip speed 1110, and/or primary sparger volumetric mass transfer coefficient (kLa) 1112 can be displayed over the course of multiple days, allowing the user to track the information for each process parameter separately.

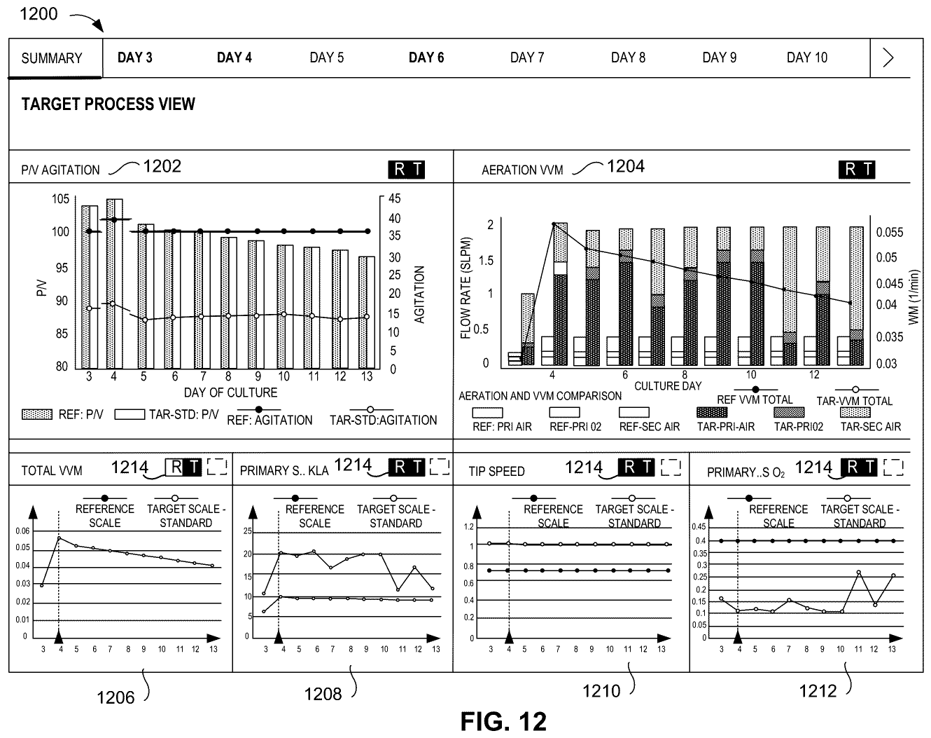

FIG. 12 illustrates an example target process view 1200, including a user-based option to show results associated with the target scale, the reference scale, and/or a combination of both the target scale and the reference scale. In the example of FIG. 12, the data can be visualized in the form of a summary and/or based on individual day(s) of interest. An example target process view includes an example graphical representation of P/V agitation 1202, an example graphical representation of aeration VVM 1204, an example graphical representation of total VVM 1206, an example graphical representation of primary sparger kLa 1208, an example graphical representation of tip speed 1210, and/or an example graphical representation of primary sparger oxygenation 1212. In the example of FIG. 12, a user has an option to view graphical data for a reference bioreactor and/or a target bioreactor (e.g., using a reference scale, using a target scale, etc.) using example scale selector 1214. For example, a comparison of the data for the reference and/or target scales can be visualized and/or quantified. In some examples, the various graphical representations can be combined on the same graph to compare changes over time using various bioprocess parameters.

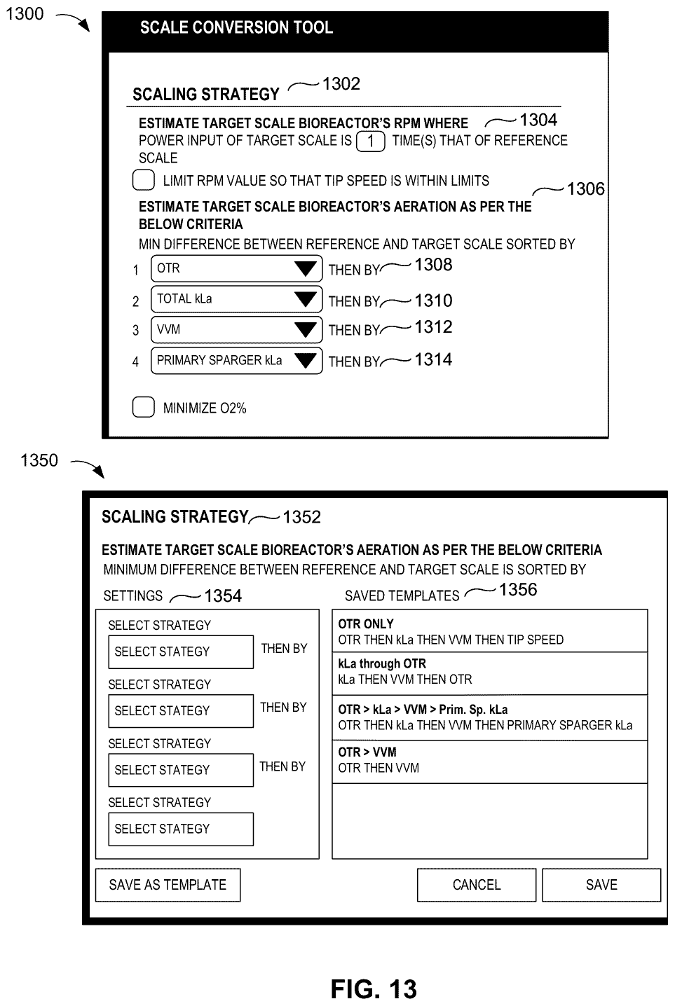

FIG. 13 illustrates example scale conversion tool(s) 1300, 1350 and example scaling strategy option(s) 1302, 1352 to reduce differences between the reference scale and the target scale. The scale conversion tool 1300 includes an example setting 1304 for a target scale bioreactor's setting (e.g., rotations per minute, RPM) where a power input of the target scale can be set to a specific value (e.g., x times that of the reference scale). In some examples, the scaling strategy 1302 can include an example option 1306 to limit a specific value (e.g., RPM) so that tip speed is within a given limit. In some examples, the scaling strategy 1302 can include an example estimation of a target scale bioreactor's aeration 1306. For example, the estimation can be based on specific criteria based on a sorting performed to reach a reduced difference between a reference scale and a target scale. The specific criteria can include identification of specific parameters that should be considered in a particular order of priority, including an example oxygen transfer rate (OTR) parameter 1308, an example total kLa parameter 1310, an example bioreactor working volume (VVM) parameter 1312, and/or an example primary sparger kLa parameter 1314. While an example order of the given parameters is presented in connection with FIG. 13, any other order can be established for reducing a difference between the reference scale and the target scale. Additionally, scaling strategy option 1352 illustrates another example of strategy selection using example settings 1354 and/or example saved template(s) 1356. In the example of FIG. 13, the settings 1354 permit a user to select a specific scaling strategy, with saved template(s) 1356 available for user selection (e.g., OTR only, kLa through OTR, OTR>kLa>VVM>primary sparger kLa, and/or OTR>VVM). As such, the templates can be used to identify which parameters should be prioritized for reducing a difference between the reference scale and the target scale.

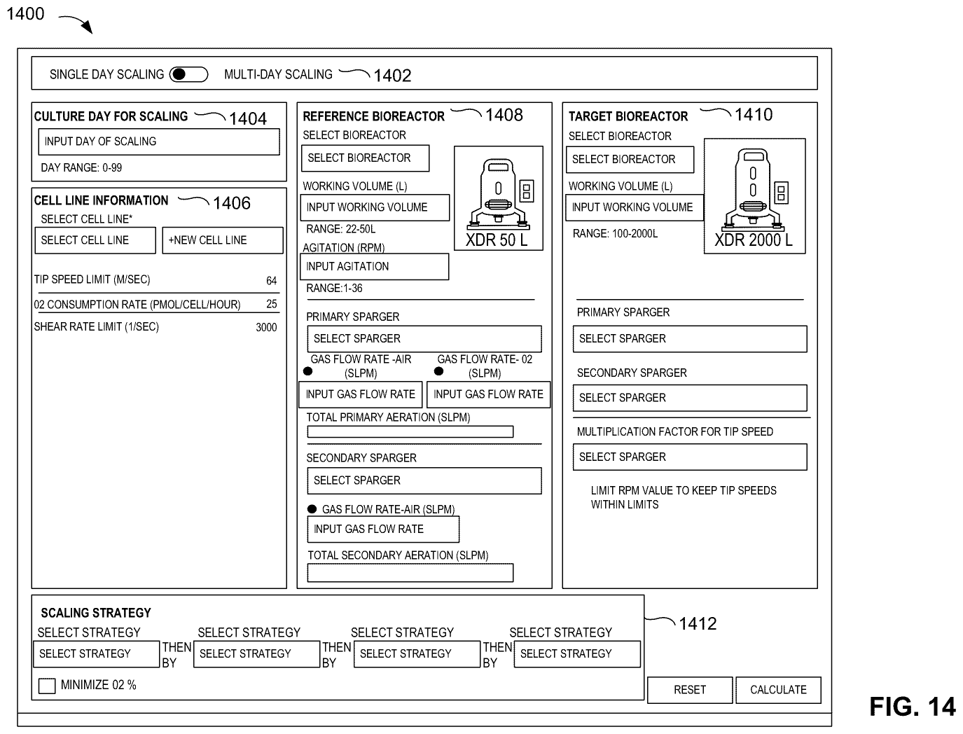

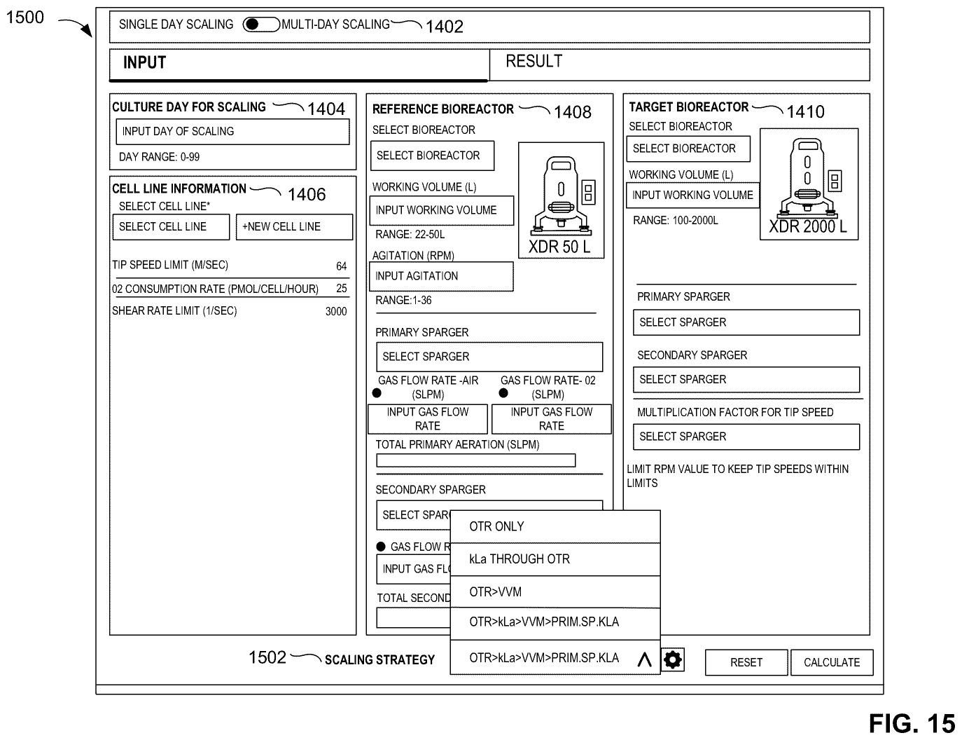

FIG. 14 illustrates an example single day scaling and/or multi-day scaling display 1400 for viewing cell line information, reference and/or target bioreactor information, and/or the corresponding scaling strategy. In the example of FIG. 14, a user can toggle between a single day scaling and/or a multi-day scaling using the example day scaling selector 1402. In some examples, the display information can include example culture day for scaling 1404, example cell line information 1406, example reference bioreactor information 1408, example target bioreactor information 1410, and/or example scaling strategy information 1412. For example, the culture day for scaling 1404 includes a user-based selection for an input to provide a day of interest corresponding to the scaling information (e.g., a range of days from 0 to 99 days). The cell line information 1406 provides for the selection of a cell line and/or addition of a new cell line, with additional information provided regarding the specific parameter settings relating to the selected cell line (e.g., tip speed limit, oxygen consumption rate, shear rate limit, etc.). In some examples, reference bioreactor information 1408 can include bioreactor selection, input for a working volume (e.g., 22-50L), agitation input (e.g., 1-36 RPM). Additional information can be provided regarding the primary sparger and/or secondary sparger, including gas flow rate(s), and/or total primary aeration. Likewise, target bioreactor information 1410 can include bioreactor selection, working volume identification (e.g., 100-2000L), as well as information related to the primary and/or secondary sparger(s). The scaling strategy information 1412 can include user-based strategy selection to determine which parameters to prioritize when reducing a difference between the reference scale and the target scale. FIG. 15 illustrates an example selection of the scaling strategy options 1412 illustrated in FIG. 14. In the example of FIG. 15, an example scaling strategy selection 1502 can include the scaling strategies described in connection with FIGS. 13-14. As such, a user can identify the scaling strategy based on specific parameters of interest. In some examples, the scaling strategy can also be set automatically using the bioprocess scaler 214 of FIG. 3.

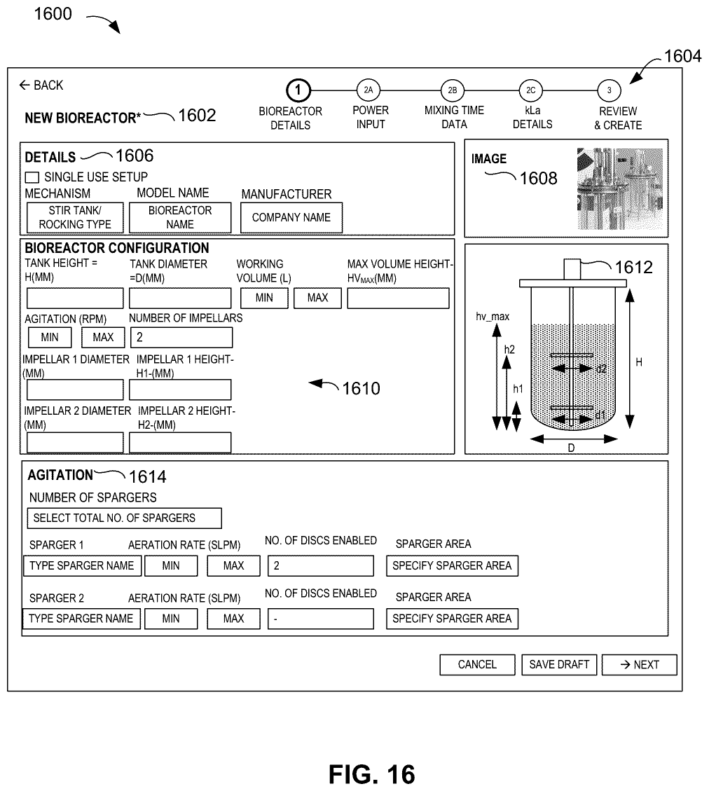

FIG. 16 illustrates an example bioreactor detail user interface 1600, including bioreactor information, configuration, and/or agitation selections. In the example of FIG. 16, the user interface 1600 provides a user with the option to enter information related to an example bioreactor of interest 1602. Part of the user interface 1600 includes an example step identifier 1604 to indicate to the user which part of the bioprocess scaling step the user is currently accessing. For example, the step identifier 1604 can include bioreactor details, power input, mixing time data, kLa details, and/or a review and create summary page. In the example of FIG. 16, the bioreactor detail user interface 1600 includes example details 1606 related to a bioreactor mechanism, model name, and/or manufacturer. In some examples, the bioreactor detail user interface 1600 includes a bioreactor configuration section 1610 related to bioreactor-based information input such as tank height, tank diameter, working volume, agitation, number of impellers, impeller diameter, and/or impeller height. An example bioreactor diagram 1612 can be used to identify specific bioreactor-based specifications. In the example of FIG. 16, the bioreactor detail user interface 1600 further includes an example agitation section 1614 to provide information related to a total number of spargers, including aeration rate(s), sparger area(s), etc. However, the bioreactor user interface 1600 is not limited to the information displayed in the example of FIG. 16.

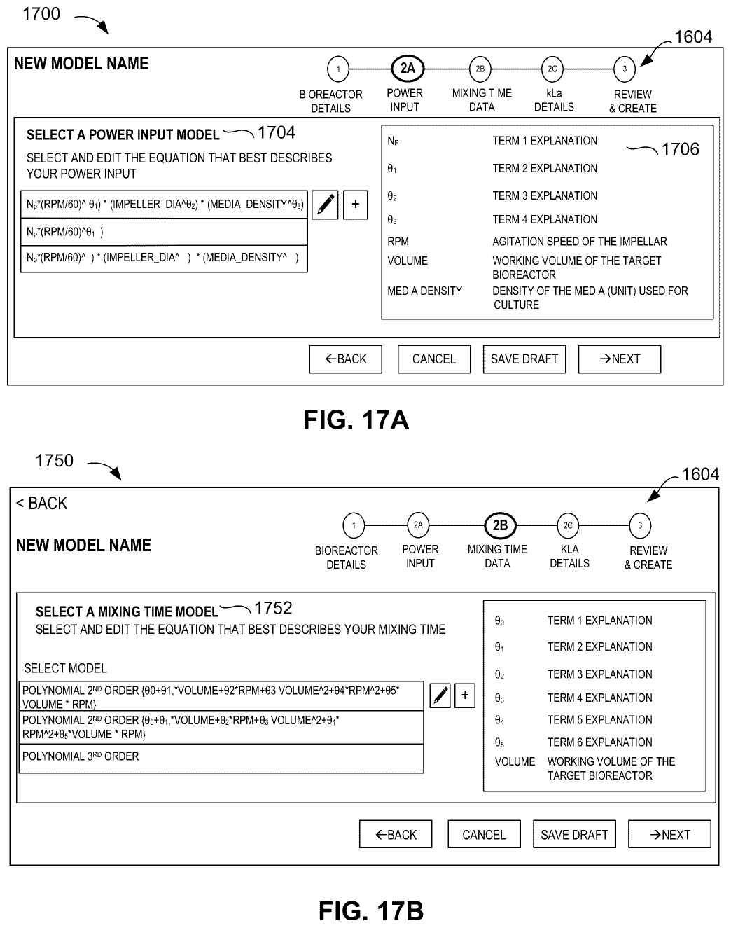

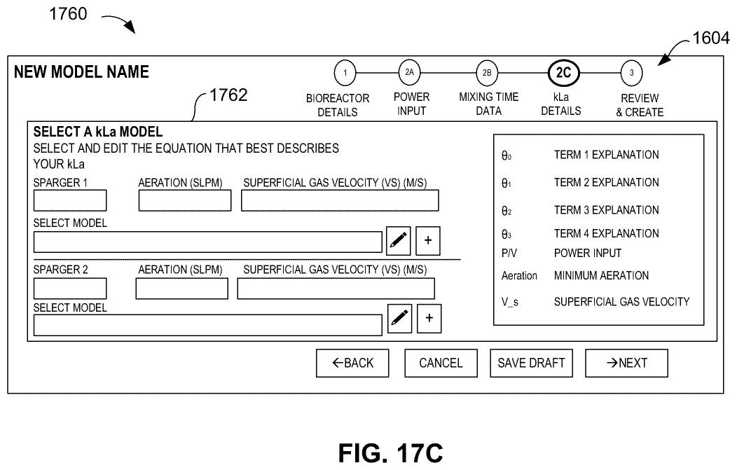

FIG. 17A illustrates an example power input user interface 1700, including selection of a power input model. In the example of FIG. 17A, the step identifier 1604 of FIG. 16 is included to indicate to the user that this user interface relates to the power input. In the example of FIG. 17A, the power input includes an example select a power input model section 1704 and/or an example parameter explanation section 1706. The select a power input model section 1704 permits a user to select and/or edit an equation that best describes an intended power input. Once the power input model section 1704 is completed, the user can progress to the mixing time information entry. FIG. 17B illustrates example mixing time data user interface 1750, including selection of a mixing time model. In the example of FIG. 17B, the step identifier 1604 of FIG. 16 is included to indicate to the user that this user interface relates to the mixing time data. In the example of FIG. 17B, the mixing time data user interface 1750 includes an example select a mixing time model section 1752. The mixing time model section 1752 permits a user to select and/or edit an equation that best described the intended mixing time. Once the mixing time model section 1752 is completed, the user can progress to the kLa details information entry. FIG. 17C illustrates example volumetric mass transfer coefficient (kLa) user interface 1760, including selection of a kLa model. In the example of FIG. 17C, the step identifier 1604 of FIG. 16 is included to indicate to the user that this user interface relates to kLa details. In some examples, the kLa user interface 1760 includes an example kLa model section 1762, allowing the user to provide information related to equations that best describe the kLa model. For example, the user can provide information related to aeration and/or superficial gas velocity.

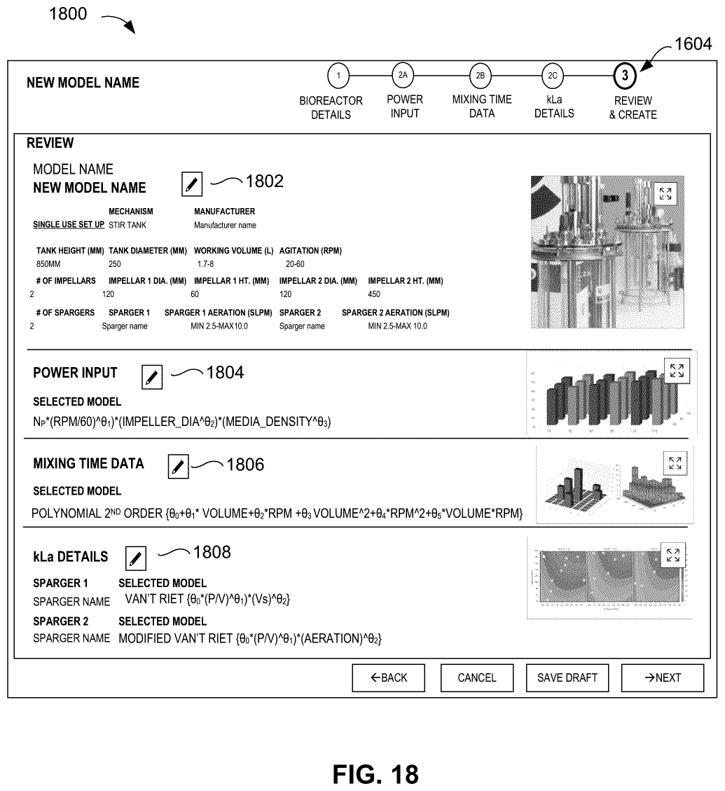

FIG. 18 illustrates an example review and create user interface 1800, including a summary of the selections shown in FIGS. 16, 17A, 17B, and/or 17C. In the example of FIG. 18, the step identifier 1604 of FIG. 16 is included to indicate to the user that this user interface relates to the review and creating of the final model. For example, the review and create user interface 1800 can include a model review section 1802, a power input review section 1804, a mixing time data review section 1806, and/or a kLa details review section 1808. As such, the review section(s) 1802, 1804, 1806, and/or 1808 summarize the information provided as part of the inputs received in the user interface(s) 1600, 1700, 1750, and/or 1760. A user can review the provided information and make any necessary corrections prior to initiating the model(s).

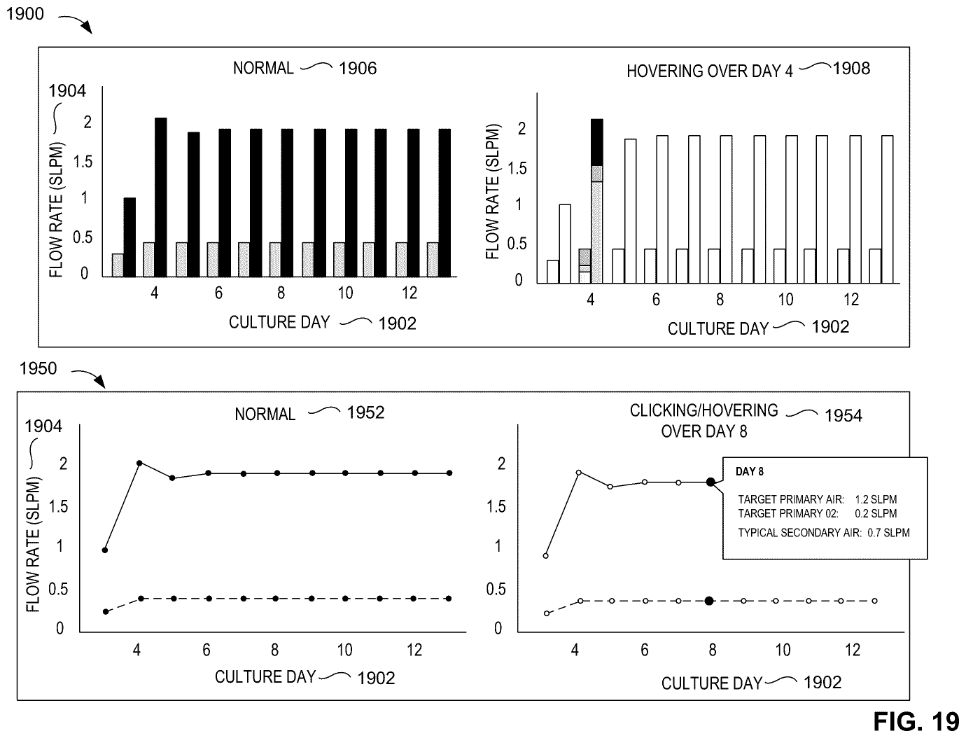

FIG. 19 illustrates an example user interface 1900 showing results that can be viewed interactively based on the selection of a particular day of interest. In the example of FIG. 19, the results can include information relating to example flow rate(s) 1904 for example cell culture day(s) 1902. In some examples, a user can view the information for the entire set of culture day(s) associated with the flow rate date using an example normal view 1906. However, the user interface 1900 allows a user to interactively view the data by hovering over a specific day of interest, as shown using an example day selection view 1908. Likewise, the provided data can be viewed in various formats, including the example line graph view 1950. In the example line graph view 1950, a user can view the data using an example normal view 1952 that shows the flow rates 1904 over the entire range of culture day(s) 1902. Likewise, a user can select a particular day of interest and view an example day selection view 1954. In some examples, the day selection view(s) 1908, 1954 can provide additional data related to the flow rate(s) 1904 (e.g., target primary air values, target primary oxygenation values, and/or typical secondary air values, etc.).

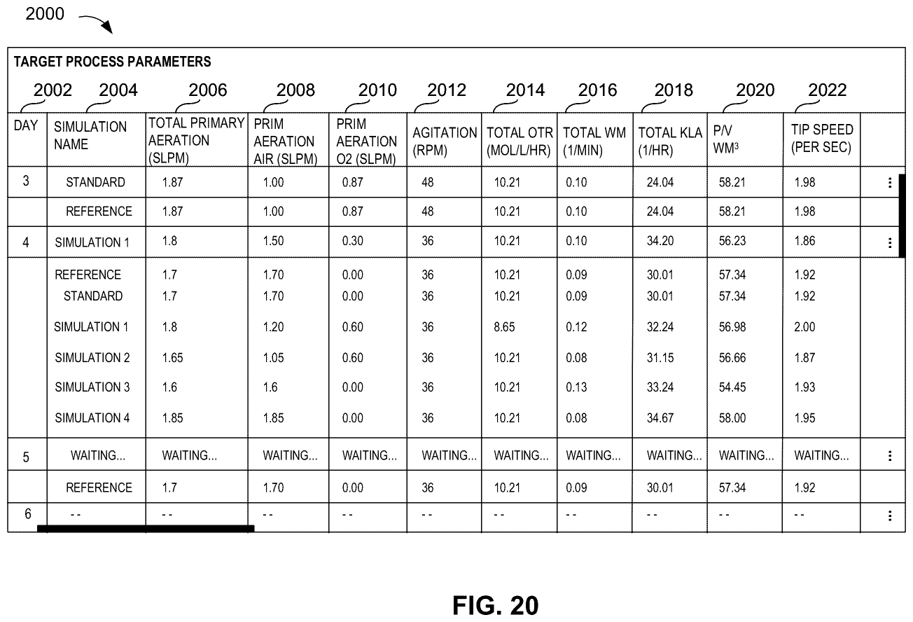

FIG. 20 illustrates an example user interface 2000 showing target process parameter data generation. The target process parameter data can include example information related to the cell culture day 2002, example simulation name 2004, example total primary aeration 2006, example primary aeration (air) 2008, example primary aeration (oxygen) 2010, example agitation 2012, example total oxygen transfer rate (OTR) 2014, example total working volume (VVM) 2016, example total kLa 2018, example power consumption per liquid volume (P/V) 2020, and/or example tip speed 2022. This information allows an overview of the target process parameters associated with a particular model.

FIG. 21 is a block diagram of an example processing platform structured to execute the example instructions of FIGS. 4-5 to implement the example bioprocess scaler of FIGS. 2 and 3. The processor platform 2100 can be a server, a personal computer, a workstation, a self-learning machine (e.g., a neural network), or any other type of computing device.

The processor platform 2100 of the illustrated example includes a processor 2112. The processor 2112 of the illustrated example is hardware. For example, the processor 2112 can be implemented by one or more integrated circuits, logic circuits, microprocessors, GPUs, DSPs, programmable logic controllers, or any other controllers from any desired family or manufacturer. The hardware processor may be a semiconductor based (e.g., silicon based) device. In this example, the processor 2112 implements the example design space generator 301, the example bioreactor value determiner 302, the example set point identifier 304, the example target bioreactor operating parameter identifier 306, the example range identifier 308, the example viable cell identifier 310, and/or the example alert manager 312.

The processor 2112 of the illustrated example includes a local memory 2113 (e.g., a cache). The processor 2112 of the illustrated example is in communication with a main memory including a volatile memory 2114 and a non-volatile memory 2116 via a bus 2118. The volatile memory 2114 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 2116 may be implemented by flash memory and/or any other desired type of memory device. Access to the main memory 2114, 2116 is controlled by a memory controller.

The processor platform 2100 of the illustrated example also includes an interface circuit 2120. The interface circuit 2120 may be implemented by any type of interface standard, such as an Ethernet interface, a universal serial bus (USB), a Bluetooth® interface, a near field communication (NFC) interface, and/or a PCI express interface.

In the illustrated example, one or more input devices 2122 are connected to the interface circuit 2120. The input device(s) 2122 permit(s) a user to enter data and/or commands into the processor 2112. The input device(s) 2122 can be implemented by, for example, an audio sensor, a microphone, a camera (still or video), a keyboard, a button, a mouse, a touchscreen, a track-pad, a trackball, isopoint and/or a voice recognition system.

One or more output devices 2124 are also connected to the interface circuit 2120 of the illustrated example. The output devices 2124 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 2120 of the illustrated example, thus, typically includes a graphics driver card, a graphics driver chip and/or a graphics driver processor.

The interface circuit 2120 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 2126. The communication can be via, for example, an Ethernet connection, a digital subscriber line (DSL) connection, a telephone line connection, a coaxial cable system, a satellite system, a line-of-site wireless system, a cellular telephone system, etc.

The processor platform 2100 of the illustrated example also includes one or more mass storage devices 2128 for storing software and/or data. Examples of such mass storage devices 2128 include floppy disk drives, hard drive disks, compact disk drives, Blu-ray disk drives, redundant array of independent disks (RAID) systems, and digital versatile disk (DVD) drives.

The machine executable instructions 2132 of FIGS. 4-5 may be stored in the mass storage device 2128, in the volatile memory 2114, in the non-volatile memory 2116, and/or on a removable non-transitory computer readable storage medium such as a CD or DVD.

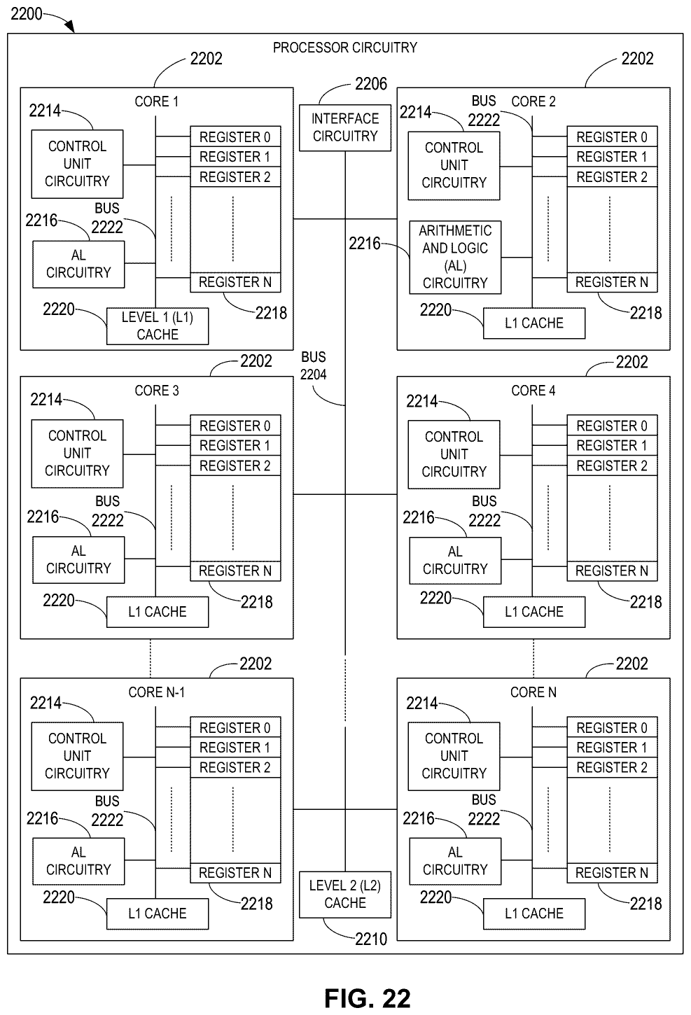

FIG. 22 is a block diagram of an example implementation of the processor circuitry 2112 of FIG. 21. In this example, the processor circuitry 2112 of FIG. 11 is implemented by a microprocessor 2200. For example, the microprocessor 2200 may implement multi-core hardware circuitry such as a CPU, a DSP, a GPU, an XPU, etc. Although it may include any number of example cores 2202 (e.g., 1 core), the microprocessor 2200 of this example is a multi-core semiconductor device including N cores. The cores 2202 of the microprocessor 2200 may operate independently or may cooperate to execute machine readable instructions. For example, machine code corresponding to a firmware program, an embedded software program, or a software program may be executed by one of the cores 2202 or may be executed by multiple ones of the cores 2202 at the same or different times. In some examples, the machine code corresponding to the firmware program, the embedded software program, or the software program is split into threads and executed in parallel by two or more of the cores 2202. The software program may correspond to a portion or all of the machine readable instructions and/or operations represented by the flowcharts of FIGS. 4-5.

The cores 2202 may communicate by an example bus 2204. In some examples, the bus 2204 may implement a communication bus to effectuate communication associated with one(s) of the cores 2202. For example, the bus 2204 may implement at least one of an Inter-Integrated Circuit (I2C) bus, a Serial Peripheral Interface (SPI) bus, a PCI bus, or a PCIe bus. Additionally or alternatively, the bus 2204 may implement any other type of computing or electrical bus. The cores 2202 may obtain data, instructions, and/or signals from one or more external devices by example interface circuitry 2206. The cores 2202 may output data, instructions, and/or signals to the one or more external devices by the interface circuitry 2206. Although the cores 2202 of this example include example local memory 2220 (e.g., Level 1 (L1) cache that may be split into an L1 data cache and an L1 instruction cache), the microprocessor 2200 also includes example shared memory 2210 that may be shared by the cores (e.g., Level 2 (L2_cache)) for high-speed access to data and/or instructions. Data and/or instructions may be transferred (e.g., shared) by writing to and/or reading from the shared memory 2210. The local memory 2220 of each of the cores 2202 and the shared memory 2210 may be part of a hierarchy of storage devices including multiple levels of cache memory and the main memory (e.g., the main memory 2114, 2116 of FIG. 21). Typically, higher levels of memory in the hierarchy exhibit lower access time and have smaller storage capacity than lower levels of memory. Changes in the various levels of the cache hierarchy are managed (e.g., coordinated) by a cache coherency policy.

Each core 2202 may be referred to as a CPU, DSP, GPU, etc., or any other type of hardware circuitry. Each core 2202 includes control unit circuitry 2214, arithmetic and logic (AL) circuitry (sometimes referred to as an ALU) 2216, a plurality of registers 2218, the L1 cache 2220, and an example bus 2222. Other structures may be present. For example, each core 2202 may include vector unit circuitry, single instruction multiple data (SIMD) unit circuitry, load/store unit (LSU) circuitry, branch/jump unit circuitry, floating-point unit (FPU) circuitry, etc. The control unit circuitry 2214 includes semiconductor-based circuits structured to control (e.g., coordinate) data movement within the corresponding core 2202. The AL circuitry 2216 includes semiconductor-based circuits structured to perform one or more mathematic and/or logic operations on the data within the corresponding core 2202. The AL circuitry 2216 of some examples performs integer based operations. In other examples, the AL circuitry 2216 also performs floating point operations. In yet other examples, the AL circuitry 2216 may include first AL circuitry that performs integer based operations and second AL circuitry that performs floating point operations. In some examples, the AL circuitry 2216 may be referred to as an Arithmetic Logic Unit (ALU). The registers 2218 are semiconductor-based structures to store data and/or instructions such as results of one or more of the operations performed by the AL circuitry 2216 of the corresponding core 2202. For example, the registers 2218 may include vector register(s), SIMD register(s), general purpose register(s), flag register(s), segment register(s), machine specific register(s), instruction pointer register(s), control register(s), debug register(s), memory management register(s), machine check register(s), etc. The registers 2218 may be arranged in a bank as shown in FIG. 22. Alternatively, the registers 2218 may be organized in any other arrangement, format, or structure including distributed throughout the core 2202 to shorten access time. The bus 2220 may implement at least one of an I2C bus, a SPI bus, a PCI bus, or a PCIe bus.

Each core 2202 and/or, more generally, the microprocessor 2200 may include additional and/or alternate structures to those shown and described above. For example, one or more clock circuits, one or more power supplies, one or more power gates, one or more cache home agents (CHAs), one or more converged/common mesh stops (CMSs), one or more shifters (e.g., barrel shifter(s)) and/or other circuitry may be present. The microprocessor 2200 is a semiconductor device fabricated to include many transistors interconnected to implement the structures described above in one or more integrated circuits (ICs) contained in one or more packages. The processor circuitry may include and/or cooperate with one or more accelerators. In some examples, accelerators are implemented by logic circuitry to perform certain tasks more quickly and/or efficiently than can be done by a general purpose processor. Examples of accelerators include ASICs and FPGAs such as those discussed herein. A GPU or other programmable device can also be an accelerator. Accelerators may be on-board the processor circuitry, in the same chip package as the processor circuitry and/or in one or more separate packages from the processor circuitry.

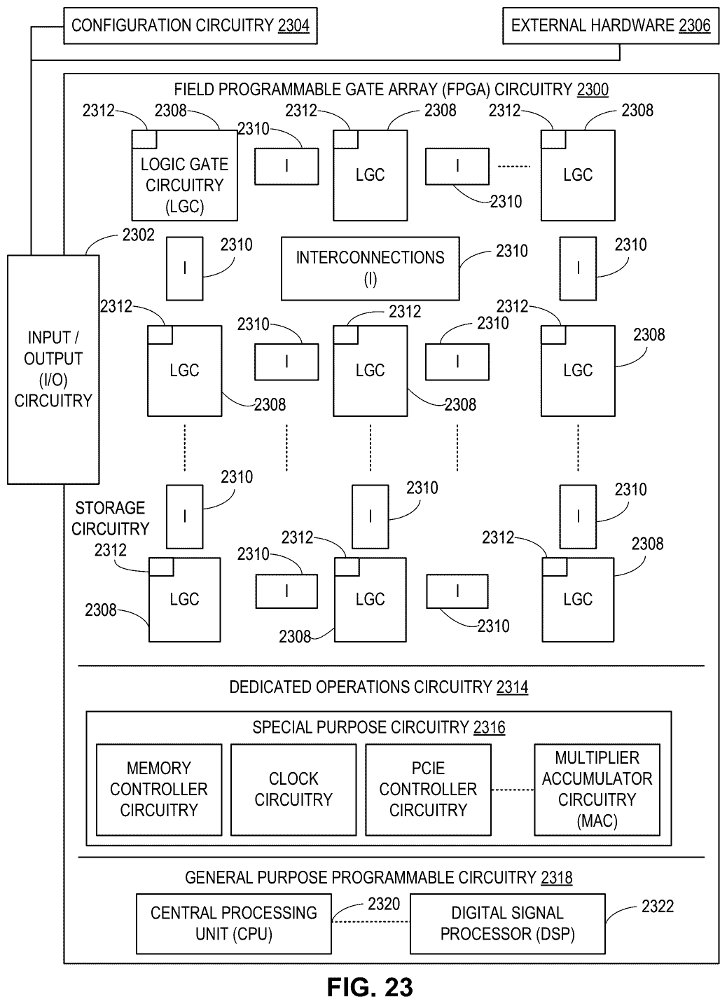

FIG. 23 is a block diagram of another example implementation of the processor circuitry 2112 of FIG. 21. In this example, the processor circuitry 2112 is implemented by FPGA circuitry 2300. The FPGA circuitry 2300 can be used, for example, to perform operations that could otherwise be performed by the example microprocessor 2200 of FIG. 22 executing corresponding machine readable instructions. However, once configured, the FPGA circuitry 2300 instantiates the machine readable instructions in hardware and, thus, can often execute the operations faster than they could be performed by a general purpose microprocessor executing the corresponding software.

More specifically, in contrast to the microprocessor 2200 of FIG. 22 described above (which is a general purpose device that may be programmed to execute some or all of the machine readable instructions represented by the flowcharts of FIGS. 4-5 but whose interconnections and logic circuitry are fixed once fabricated), the FPGA circuitry 2300 of the example of FIG. 23 includes interconnections and logic circuitry that may be configured and/or interconnected in different ways after fabrication to instantiate, for example, some or all of the machine readable instructions represented by the flowcharts of FIGS. 4-5. In particular, the FPGA 2300 may be thought of as an array of logic gates, interconnections, and switches. The switches can be programmed to change how the logic gates are interconnected by the interconnections, effectively forming one or more dedicated logic circuits (unless and until the FPGA circuitry 2300 is reprogrammed). The configured logic circuits enable the logic gates to cooperate in different ways to perform different operations on data received by input circuitry. Those operations may correspond to some or all of the software represented by the flowcharts of FIGS. 4-5. As such, the FPGA circuitry 2300 may be structured to effectively instantiate some or all of the machine readable instructions of the flowcharts of FIGS. 4-5 as dedicated logic circuits to perform the operations corresponding to those software instructions in a dedicated manner analogous to an ASIC. Therefore, the FPGA circuitry 2300 may perform the operations corresponding to the some or all of the machine readable instructions of FIGS. 4-5 faster than the general purpose microprocessor can execute the same.

In the example of FIG. 23, the FPGA circuitry 2300 is structured to be programmed (and/or reprogrammed one or more times) by an end user by a hardware description language (HDL) such as Verilog. The FPGA circuitry 2300 of FIG. 23, includes example input/output (I/O) circuitry 2302 to obtain and/or output data to/from example configuration circuitry 2304 and/or external hardware (e.g., external hardware circuitry) 2306. For example, the configuration circuitry 2304 may implement interface circuitry that may obtain machine readable instructions to configure the FPGA circuitry 2300, or portion(s) thereof. In some such examples, the configuration circuitry 2304 may obtain the machine readable instructions from a user, a machine (e.g., hardware circuitry (e.g., programmed or dedicated circuitry) that may implement an Artificial Intelligence/Machine Learning (AI/ML) model to generate the instructions), etc. In some examples, the external hardware 2306 may implement the microprocessor 2200 of FIG. 22. The FPGA circuitry 2300 also includes an array of example logic gate circuitry 2308, a plurality of example configurable interconnections 2310, and example storage circuitry 2312. The logic gate circuitry 2308 and interconnections 2310 are configurable to instantiate one or more operations that may correspond to at least some of the machine readable instructions of FIGS. 4-5 and/or other desired operations. The logic gate circuitry 2308 shown in FIG. 23 is fabricated in groups or blocks. Each block includes semiconductor-based electrical structures that may be configured into logic circuits. In some examples, the electrical structures include logic gates (e.g., And gates, Or gates, Nor gates, etc.) that provide basic building blocks for logic circuits. Electrically controllable switches (e.g., transistors) are present within each of the logic gate circuitry 2308 to enable configuration of the electrical structures and/or the logic gates to form circuits to perform desired operations. The logic gate circuitry 2308 may include other electrical structures such as look-up tables (LUTs), registers (e.g., flip-flops or latches), multiplexers, etc.

The interconnections 2310 of the illustrated example are conductive pathways, traces, vias, or the like that may include electrically controllable switches (e.g., transistors) whose state can be changed by programming (e.g., using an HDL instruction language) to activate or deactivate one or more connections between one or more of the logic gate circuitry 2308 to program desired logic circuits.

The storage circuitry 2312 of the illustrated example is structured to store result(s) of the one or more of the operations performed by corresponding logic gates. The storage circuitry 2312 may be implemented by registers or the like. In the illustrated example, the storage circuitry 2312 is distributed amongst the logic gate circuitry 2308 to facilitate access and increase execution speed.

The example FPGA circuitry 2300 of FIG. 23 also includes example Dedicated Operations Circuitry 2314. In this example, the Dedicated Operations Circuitry 2314 includes special purpose circuitry 2316 that may be invoked to implement commonly used functions to avoid the need to program those functions in the field. Examples of such special purpose circuitry 2316 include memory (e.g., DRAM) controller circuitry, PCIe controller circuitry, clock circuitry, transceiver circuitry, memory, and multiplier-accumulator circuitry. Other types of special purpose circuitry may be present. In some examples, the FPGA circuitry 2300 may also include example general purpose programmable circuitry 2318 such as an example CPU 2320 and/or an example DSP 2322. Other general purpose programmable circuitry 2318 may additionally or alternatively be present such as a GPU, an XPU, etc., that can be programmed to perform other operations.

Although FIGS. 22 and 23 illustrate two example implementations of the processor circuitry 2112 of FIG. 21, many other approaches are contemplated. For example, as mentioned above, modern FPGA circuitry may include an on-board CPU, such as one or more of the example CPU 2320 of FIG. 23. Therefore, the processor circuitry 2112 of FIG. 21 may additionally be implemented by combining the example microprocessor 2200 of FIG. 22 and the example FPGA circuitry 2300 of FIG. 23. In some such hybrid examples, a first portion of the machine readable instructions represented by the flowcharts of FIGS. 4-5 may be executed by one or more of the cores 2202 of FIG. 22 and a second portion of the machine readable instructions represented by the flowcharts of FIG. 4-5 may be executed by the FPGA circuitry 2300 of FIG. 23.

In some examples, the processor circuitry 2112 of FIG. 21 may be in one or more packages. For example, the processor circuitry 2100 of FIG. 21 and/or the FPGA circuitry 2300 of FIG. 23 may be in one or more packages. In some examples, an XPU may be implemented by the processor circuitry 2112 of FIG. 21, which may be in one or more packages. For example, the XPU may include a CPU in one package, a DSP in another package, a GPU in yet another package, and an FPGA in still yet another package.

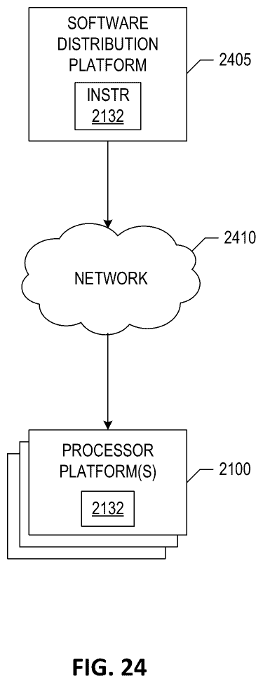

A block diagram illustrating an example software distribution platform 2405 to distribute software such as the example machine readable instructions 2132 of FIG. 21 to hardware devices owned and/or operated by third parties is illustrated in FIG. 24. The example software distribution platform 2405 may be implemented by any computer server, data facility, cloud service, etc., capable of storing and transmitting software to other computing devices. The third parties may be customers of the entity owning and/or operating the software distribution platform 2405. For example, the entity that owns and/or operates the software distribution platform 2405 may be a developer, a seller, and/or a licensor of software such as the example machine readable instructions 2132 of FIG. 21. The third parties may be consumers, users, retailers, OEMs, etc., who purchase and/or license the software for use and/or re-sale and/or sub-licensing. In the illustrated example, the software distribution platform 2405 includes one or more servers and one or more storage devices. The storage devices store the machine readable instructions 2132, which may correspond to the example machine readable instructions of FIGS. 4-5, as described above. The one or more servers of the example software distribution platform 2405 are in communication with a network 2410, which may correspond to any one or more of the Internet and/or any of the example networks described above. In some examples, the one or more servers are responsive to requests to transmit the software to a requesting party as part of a commercial transaction. Payment for the delivery, sale, and/or license of the software may be handled by the one or more servers of the software distribution platform and/or by a third party payment entity. The servers enable purchasers and/or licensors to download the machine readable instructions 2132 from the software distribution platform 2405. For example, the software, which may correspond to the example machine readable instructions of FIGS. 4-5, may be downloaded to the example processor platform 2100, which is to execute the machine readable instructions 2132 to implement the bioprocess scaler 214 of FIGS. 2-3. In some example, one or more servers of the software distribution platform 2405 periodically offer, transmit, and/or force updates to the software (e.g., the example machine readable instructions 2132 of FIG. 21) to ensure improvements, patches, updates, etc., are distributed and applied to the software at the end user devices.

From the foregoing, it will be appreciated that the above disclosed methods, apparatus, and articles of manufacture permit the identification of a range of potential changes in bioprocess variables that can be optimized while staying within a given design space. In some examples, a user navigates the expanded design space and identifies settings that will support a given bioprocess. As such, the identified design space gives the user flexibility to navigate inside the design space as well as permit improved investigation of process deviations. Methods and apparatus disclosed herein allow a user to readily access results when scaling with different bioreactor configurations, as well as easily compare results according to various criteria. Examples disclosed herein permit the identification of acceptable variable value ranges for a target bioreactor (e.g., agitation, aeration) based on a specific target bioreactor configuration, cell culture information, identification of reference bioreactor-based variable values (e.g., tip speed, mixing time, etc.) and/or the identification of set points that reduce deviations from reference bioreactor-based variable values. Additionally, method and apparatus disclosed herein introduce a simulation functionality that gives a user the ability to explore a design space and find other settings that maintain desired criteria for the bioprocess.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. An apparatus for bioprocess scaling, the apparatus comprising:
at least one memory to store instructions; and
processor circuitry to execute the instructions to:
identify an operating parameter of a target bioreactor;
determine an upper boundary or a lower boundary defining a design space for at least one bioreactor process parameter to match at least one of a first target parameter range or a second target parameter range based on the operating parameter;
simulate changes in the first target parameter range or a second target parameter range based on an adjustment to the upper boundary or the lower boundary in the design space; and
configure the target bioreactor using output obtained from the adjustment to the upper boundary or the lower boundary to identify a match between the first target parameter range or the second target parameter range and a user-based input of a target bioprocess parameter value.

2. The apparatus of claim 1, wherein the first target parameter is an oxygen transfer rate (OTR) or a bioreactor working volume.

3. The apparatus of claim 1, wherein the processor circuitry is to identify at least one bioreactor process parameter for scaling from a reference scale to a target scale.

4. The apparatus of claim 3, wherein the scaling includes simulating process parameter adjustments to determine a target bioreactor value range that reduces deviations from the reference scale.

5. The apparatus of claim 3, wherein the scaling includes adjustment of a scaling parameter, the scaling parameter including a tip speed, an energy dissipation rate, a mixing time, a mass transfer coefficient, or a shear force.

6. The apparatus of claim 3, wherein the processor circuitry is to determine a primary or a secondary sparger mass transfer coefficient.

7. The apparatus of claim 6, wherein the processor circuitry is to calculate viable cell density (VCD) based on primary aeration or secondary aeration associated with the primary sparger or the secondary sparger.

8. A method for bioprocess scaling, the method comprising:
identifying an operating parameter of a target bioreactor;
determining an upper boundary or a lower boundary for at least one bioreactor process parameter to match at least one of a first target parameter range or a second target parameter range based on the operating parameter;
simulating changes in the first target parameter range or a second target parameter range based on an adjustment to the upper boundary or the lower boundary; and
configuring the target bioreactor using output obtained from the adjustment to the upper boundary or the lower boundary to identify a match between the first target parameter range or the second target parameter range and a user-based input of a target bioprocess parameter value.

9. The method of claim 8, wherein the first target parameter is an oxygen transfer rate (OTR) or a bioreactor working volume.

10. The method of claim 8, further including identifying at least one bioreactor process parameter for scaling from a reference scale to a target scale.

11. The method of claim 10, further including simulating process parameter adjustments to determine a target bioreactor value range that reduces deviations from the reference scale.

12. The method of claim 10, further including adjusting a scaling parameter, the scaling parameter including a tip speed, an energy dissipation rate, a mixing time, a mass transfer coefficient, or a shear force.

13. The method of claim 10, further including determining a primary or a secondary sparger mass transfer coefficient.

14. The method of claim 13, further including calculating viable cell density (VCD) based on primary aeration or secondary aeration associated with the primary sparger or the secondary sparger.

15. At least one computer readable storage medium comprising instructions that, when executed, cause at least one processor to at least:
identify an operating parameter of a target bioreactor;
determine an upper boundary or a lower boundary for at least one bioreactor process parameter to match at least one of a first target parameter range or a second target parameter range based on the operating parameter;
simulate changes in the first target parameter range or a second target parameter range based on an adjustment to the upper boundary or the lower boundary; and
configure the target bioreactor using output obtained from the adjustment to the upper boundary or the lower boundary to identify a match between the first target parameter range or the second target parameter range and a user-based input of a target bioprocess parameter value.

16. The at least one storage medium as defined in claim 15, wherein the computer readable instructions, when executed, cause the one or more processors to identify at least one bioreactor process parameter for scaling from a reference scale to a target scale.

17. The at least one storage medium as defined in claim 16, wherein the computer readable instructions, when executed, cause the one or more processors to simulate process parameter adjustments to determine a target bioreactor value range that reduces deviations from the reference scale.

18. The at least one storage medium as defined in claim 16, wherein the computer readable instructions, when executed, cause the one or more processors to adjust a scaling parameter, the scaling parameter including a tip speed, an energy dissipation rate, a mixing time, a mass transfer coefficient, or a shear force.

19. The at least one storage medium as defined in claim 16, wherein the computer readable instructions, when executed, cause the one or more processors to determine a primary or a secondary sparger mass transfer coefficient.

20. The at least one storage medium as defined in claim 19, wherein the computer readable instructions, when executed, cause the one or more processors to calculate viable cell density (VCD) based on primary aeration or secondary aeration associated with the primary sparger or the secondary sparger.
